(19) United States
(12) Patent Application Publication
(10) Pub. No.: US 2023/0301536 A1
(43) Pub. Date: Sep. 28, 2023

(54) BIOLOGICAL MEASUREMENT DEVICE, BIOLOGICAL MEASUREMENT METHOD, AND NON-TRANSITORY COMPUTER-READABLE RECORDING MEDIUM

(71) Applicant: Panasonic Intellectual Property Management Co., Ltd., Osaka (JP)

(72) Inventors: HITOSHI TOMINAGA, Kyoto (JP); HIROTO YANAGAWA, Osaka (JP)

(21) Appl. No.: 18/327,928

(22) Filed: Jun. 2, 2023

Related U.S. Application Data
(63) Continuation of application No. PCT/JP2021/044432, filed on Dec. 3, 2021.

(30) Foreign Application Priority Data
Dec. 25, 2020 (JP) 2020-217155

(51) Int. Cl. A61B 5/026; A61B 5/00

(52) U.S. Cl. CPC A61B 5/0261; A61B 5/4064

(57) ABSTRACT

A biological measurement device includes a light emitting device, a sensor, and a processing circuit. The light emitting device irradiates a first region and a second region, which is located on an upper side relative to the first region, of a forehead of a subject with light. The sensor detects first scattering light generated by the light incident on the first region and second scattering light generated by the light incident on the second region and outputs detection signals according to intensities of the first and second scattering light. The processing circuit selects one of the first and second regions as a target region based on the detection signals and/or an image signal indicative of an image including a face of the subject and generates and outputs brain activity data indicative of a state of brain activity of the subject based on the detection signal in the selected target region.

### BACKGROUND

### 1. Technical Field

The present disclosure relates to a biological measurement device and others.

### 2. Description of the Related Art

Various techniques for measuring a biological signal that fluctuates in accordance with brain activity of a subject have been developed. For example, various techniques for acquiring a signal indicative of a state of a cerebral blood flow of a subject by using near infrared spectroscopy (NIRS) have been developed. Examples of such techniques are, for example, disclosed in Japanese Unexamined Patent Application Publication No. 2017-009584 (hereinafter referred to as Patent Literature 1) and International Publication No. 2012/150657 (hereinafter referred to as Patent Literature 2).

Patent Literature 1 discloses an example of a non-contact type NIRS device. The NIRS device disclosed in Patent Literature 1 generates a signal indicative of a temporal change in cerebral blood flow by repeating an operation of irradiating a forehead of a subject with light such as near-infrared light and detecting a light component scattered inside the forehead.

Patent Literature 2 discloses a technique of measuring a user's cerebral blood flow amount by using a non-contact type NIRS device and estimating a user's degree of concentration on the basis of a change in cerebral blood flow amount.

### SUMMARY

The biological measurement techniques such as the ones described above are required to further improve accuracy of measurement of a state that changes in accordance with a brain activity, such as a subject's cerebral blood flow state, psychological state, or physical state.

In one general aspect, the techniques disclosed here feature a biological measurement device including a light emitting device, a sensor, and a processing circuit. The light emitting device irradiates a first region and a second region, which is located on an upper side relative to the first region, of a forehead of a subject with light. The sensor detects first scattering light generated by the light incident on the first region and second scattering light generated by the light incident on the second region and outputs detection signals according to intensities of the first scattering light and the second scattering light. The processing circuit selects one of the first region and the second region as a target region on the basis of the detection signals and/or an image signal indicative of an image including a face of the subject and generates and outputs brain activity data indicative of a state of brain activity of the subject on the basis of the detection signal in the selected target region.

According to the technique of the present disclosure, it is possible to improve accuracy of estimation of a brain activity state of a subject.

It should be noted that general or specific aspects of the present disclosure may be implemented as a system, a device, a method, an integrated circuit, a computer program, a computer-readable recording medium, or any selective combination thereof. Examples of the computer-readable recording medium include non-volatile recording media such as a compact disc-read only memory (CD-ROM). The device may include one or more devices. In a case where the device includes two or more devices, the two or more devices may be disposed in a single apparatus or may be separately disposed in separate two or more apparatuses. In the specification and claims, the “device” can mean not only a single device, but also a system including devices.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 schematically illustrates a configuration of a biological measurement device according to a first embodiment;

FIG. 2A illustrates an example of a temporal change in intensity of an emitted light pulse Ie and temporal changes in intensity of a surface reflected component I1 and an internal scattered component I2 in a reflected light pulse;

FIG. 2B illustrates another example of the temporal change in intensity of the emitted light pulse Ie and the temporal changes in intensity of the surface reflected component I1 and the internal scattered component I2 in the reflected light pulse;

FIG. 3 illustrates an example of an outline configuration of one pixel of an image sensor;

FIG. 4 illustrates an example of a configuration of the image sensor;

FIG. 5 schematically illustrates an example of an operation performed within one frame;

FIG. 6 schematically illustrates a waveform of a light intensity of a reflected light pulse in a case where a rectangular-wave light pulse is emitted;

FIG. 7A is a timing diagram illustrating an example of an operation of detecting the internal scattered component I2;

FIG. 7B is a timing diagram illustrating an example of an operation of detecting the surface reflected component I1.

FIG. 8 is a flowchart illustrating an outline of an operation of controlling a light source and the image sensor by a control circuit;

FIG. 9A illustrates an example of regions to which brain activity data can be output;

FIG. 9B illustrates another example of regions to which brain activity data can be output;

FIG. 9C illustrates still another example of regions to which brain activity data can be output;

FIG. 9D illustrates still another example of regions to which brain activity data can be output;

FIG. 10 is a flowchart illustrating an example of processing for generating brain activity data of a user;

FIG. 11 illustrates an example of an operation of switching a target region in accordance with a change in facial expression of the user;

FIG. 12A illustrates an example of a relationship between an amount of displacement of an eyebrow and a switching period;

FIG. 12B illustrates another example of the relationship between the amount of displacement of the eyebrow and the switching period;

FIG. 12C illustrates an example of a relationship between a rate of change of the eyebrow and the switching period;

FIG. 13 is a flowchart illustrating another example of processing for deciding a target region;

FIG. 14 schematically illustrates a configuration of a biological measurement device according to a second embodiment;

FIG. 15 schematically illustrates an example of a configuration of an NIRS sensor; and

FIG. 16 is a flowchart illustrating an example of an operation of the biological measurement device according to the second embodiment.

### DETAILED DESCRIPTIONS

In the present disclosure, all or a part of any of circuit, unit, device, part or portion, or any of functional blocks in the block diagrams may be implemented as one or more of electronic circuits including a semiconductor device, a semiconductor integrated circuit (IC) or a (large scale integration (LSI). The LSI or IC can be integrated into one chip, or also can be a combination of chips. For example, functional blocks other than a memory may be integrated into one chip. The name used here is LSI or IC, but it may also be called system LSI, very large scale integration (VLSI), or ultra large scale integration (ULSI) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or a part of the functions or operations of the circuit, unit, device, part or portion are implemented by executing software. In such a case, the software is recorded on one or more non-transitory recording media such as a ROM, an optical disk or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface.

### Underlying Knowledge Forming Basis of the Present Disclosure

Underlying knowledge forming basis of the present disclosure is described before embodiments of the present disclosure are described.

In the biological measurement technology, it is known that a change in user's state during measurement influences a result of the measurement. For example, a result of measurement of a state of brain activity using near infrared spectroscopy (NIRS) includes various kinds of noise. In particular, the inventors of the present invention focused on a change in shape of user's skin during measurement that greatly influences a signal.

When a shape of skin of a head changes, for example, due to a change in facial expression during measurement, a thickness of a scalp through which light passes and an angle of a scalp surface change, and as a result, an intensity of scattering light from an inside of the scalp and an intensity of reflected light from the scalp surface fluctuate. Accordingly, when a shape of skin of the head changes during measurement, a detected signal greatly fluctuates, and it is therefore impossible to correctly estimate a state of brain activity. This problem is remarkable especially in a case where a non-contact type NIRS device is used as a measurement device.

In a case where brain activity is measured for the purpose of research, an operator of an NIRS device can recognize irregularity of a signal, notify a user that a measurement result includes an error, and prompt the user to perform measurement again. However, this is difficult, for example, in a case where a user himself or herself routinely measures a state of brain activity. It is desired that a state of brain activity can be estimated correctly even in a case where a shape of skin changes during measurement.

Patent Literature 2 discloses a method for selecting a measurement portion of relatively high sensitivity from among measurement portions in a user's brain and calculating a degree of user's concentration on the basis of a signal acquired in the selected measurement portion. The device disclosed in Patent Literature 2 calculates, for each of the measurement portions, a ratio of an amount of decrease in cerebral blood flow during a concentration period to an amount of decrease in cerebral blood flow during a resting period and calculates a degree of user's concentration by using a cerebral blood flow amount measured in a measurement portion where the ratio is largest. According to such a method, it is possible to improve accuracy of estimation of a degree of user's concentration. However, noise resulting from a change in skin shape is far larger than a difference in signal amount resulting from a difference in sensitivity among measurement portions, and therefore in a case where the noise is generated, selection of a measurement portion based on the difference in sensitivity is meaningless.

According to data obtained by experiments conducted by the inventors of the present invention, among measurement regions of a forehead, a region located on a relatively lower side is higher in sensitivity of detection of an amount of change in cerebral blood flow than a region located on a relatively upper side. Meanwhile, it was revealed that in a region located on a lower side, a shape of a surface layer of skin greatly changes due to influence of a change in facial expression, and resulting noise is large. Based on this finding, the inventors of the present invention arrived at using a signal from a lower region where sensitivity is high in a state where noise is small and using a signal from an upper region where influence of noise is small in a state where noise is large, and completed the technique of the present disclosure.

An outline of an embodiment of the present disclosure is described below.

A biological measurement device according to an exemplary embodiment of the present disclosure includes a light emitting device, a sensor, and a processing circuit. The light emitting device irradiates a first region and a second region, which is located on an upper side relative to the first region, of a forehead of a subject with light. The sensor detects first scattering light generated by the light incident on the first region and second scattering light generated by the light incident on the second region and outputs detection signals according to intensities of the first scattering light and the second scattering light. The processing circuit selects one of the first region and the second region as a target region on the basis of the detection signals and/or an image signal indicative of an image including a face of the subject and generates and outputs brain activity data indicative of a state of brain activity of the subject on the basis of the detection signal in the selected target region.

In the present disclosure, the “brain activity data” is data in any format indicative of a state of brain activity of a subject. The brain activity data can be, for example, data indicative of a state or an amount of hemoglobin in blood in the brain or data indicative of a physical state or a psychological state of the subject estimated from the state of hemoglobin in blood in the brain. Blood receives oxygen from lungs and carries oxygen to tissues, and receives carbon dioxide from tissues and circulates carbon dioxide through the lungs. 100 ml of blood contains approximately 15 g of hemoglobin. Hemoglobin bound to oxygen is called oxyhemoglobin. Hemoglobin that is not bound to oxygen is called deoxyhemoglobin. The brain activity data can include, for example, at least one concentration information among an oxyhemoglobin concentration, a deoxyhemoglobin concentration, and a total hemoglobin concentration in cerebral blood. The total hemoglobin concentration is a sum of the oxyhemoglobin concentration and the deoxyhemoglobin concentration. The brain activity data may be data indicative of a mental state related to a degree of concentration, interest, feeling, sleepiness, fatigue, or the like of a subject estimated from the concentration information. The brain activity data may be image data indicative of a spatial distribution of a state of brain activity of the subject. The brain activity data may be moving image data indicative of a temporal change of the spatial distribution of the state of brain activity of the subject.

According to the biological measurement device, brain activity data of the subject can be generated while selecting an appropriate one of the first region and the second region as the target region on the basis of at least one of the detection signals and the image signal. The first region is high in sensitivity but is more influenced by noise resulting from a change in shape of skin or the like. On the other hand, the second region that is located on an upper side is relatively low in sensitivity but is less influenced by noise resulting from a change in shape of skin or the like. The processing circuit can detect, for example, a change in shape of skin on the basis of at least one of the detection signals and the image signal, select a more appropriate one of the first region and the second region in accordance with a result of the detection, and generate and output brain activity data in the more appropriate region. By such an operation, both sensitivity and accuracy of measurement can be achieved.

The biological measurement device may be a non-contact type NIRS device or may be a contact type NIRS device. In a case where the measurement device is a non-contact type NIRS device, the light emitting device and the sensor are disposed apart from the subject. In a case where the measurement device is a contact type NIRS device, the light emitting device and the sensor are disposed close to the skin of the subject. In either case, the above effect can be obtained.

The sensor may be an image sensor that outputs the detection signals and the image signal. Use of the image sensor makes it possible to acquire detection signals within a relatively wide range including the first region and the second region at one time. Furthermore, an image signal can be acquired by the image sensor. Alternatively, the biological measurement device may include an image sensor separately from the sensor. The processing circuit can select the target region on the basis of a temporal change of the image signal.

The processing circuit may detect movement of the eyebrow of the subject included in an image indicated by the image signal and select the target region on the basis of the movement of the eyebrow. More specifically, the processing circuit may select the second region as the target region in a case where an amount of displacement of the eyebrow of the subject from a reference position is larger than a threshold value during a measurement period and select the first region as the target region in a case where the amount of displacement of the eyebrow of the subject from the reference position is not larger than the threshold value during the measurement period. By such an operation, brain activity data can be generated while selecting an appropriate one of the first region and the second region in accordance with a change in shape of skin of the forehead of the subject during measurement.

The processing circuit may select the target region on the basis of the detection signals output from the sensor. For example, the processing circuit may generate a cerebral blood flow signal indicative of a state of hemoglobin in cerebral blood in the first region on the basis of the detection signal and select the target region on the basis of a temporal change of the cerebral blood flow signal.

The cerebral blood flow signal may indicate temporal changes of an oxyhemoglobin concentration and a deoxyhemoglobin concentration in the cerebral blood of the subject. During a period of measurement by the sensor, the processing circuit may select the first region as the target region during a period where one of the oxyhemoglobin concentration and the deoxyhemoglobin concentration in the first region increases and the other one of the oxyhemoglobin concentration and the deoxyhemoglobin concentration in the first region decreases and select the second region as the target region during the period where both of the oxyhemoglobin concentration and the deoxyhemoglobin concentration in the first region increase or decrease. By such an operation, a change in signal resulting from brain activity and a change in signal resulting from a change in shape of skin can be distinguished, and brain activity data can be generated while selecting a more appropriate region.

The processing circuit may generate a cerebral blood flow signal indicative of a state of hemoglobin in cerebral blood in the first region and a cerebral blood flow signal indicative of a state of hemoglobin in cerebral blood the second region on the basis of the detection signals in the first region and the second region and generate the brain activity data on the basis of the cerebral blood flow signal in the selected target region. By such an operation, for example, brain activity data indicative of a degree of concentration or the like can be generated from the cerebral blood flow signal.

The first region and the second region may be apart from each other or a part of the first region and a part of the second region may overlap each other. An area of the first region and an area of the second region may be identical to each other or may be different from each other. For example, the area of the second region may be larger than the area of the first region. The second region is lower in sensitivity than the first region. Therefore, processing such as integrating a larger number of optical signals may be performed by setting the area of the second region larger than the area of the first region.

The light emitting device includes one or more light sources. The sensor includes one or more photodetection elements. The processing circuit generates the brain activity data on the basis of the detection signals output from the sensor. The biological measurement device may repeatedly perform the above operations of the light emitting device, the sensor, and the processing circuit, for example, on a predetermined cycle during a measurement period. In this way, brain activity data of the subject can be output periodically.

The light emitting device may irradiate the first region and the second region at different timings or may irradiate the first region and the second region concurrently. The light emitting device may separately include a light source that irradiates the first region and a light source that irradiates the second region or may include a single light source that irradiates a relatively wide range including the first region and the second region at once.

The light emitting device may be configured to emit a light pulse toward the first region and the second region. In this case, the sensor may detect the first scattering light and the second scattering light by detecting at least a part of a component after start of decrease in intensity among components of a reflected light pulse from the first region and the second region generated by emission of the light pulse. In other words, the sensor may detect the first scattering light by detecting at least a part of a component after start of decrease in intensity of a first reflected light pulse from the first region. The sensor may detect the second scattering light by detecting at least a part of a component after start of decrease in intensity of a second reflected light pulse from the second region. The “component after start of decrease in intensity” refers to a component of a reflected light pulse that reaches the sensor during at least a part of a period from start of decrease in intensity to end of decrease in intensity. The sensor may start detection of a reflected light pulse after start of decrease in intensity of the reflected light pulse. This makes it possible to increase a percentage of a component scattered inside a living body in a detected signal.

The light emitting device may include a first light source that emits a first light pulse having a first wavelength that is equal to or longer than 650 nm and shorter than 805 nm in air and a second light source that emits a second light pulse having a second wavelength that is longer than 805 nm and equal to or shorter than 950 nm in air. The sensor may be configured to detect at least a part of a component after start of decrease in intensity among components of a reflected light pulse generated by irradiation of the first region with the first light pulse and output a first signal according to an amount of the detected light, detect at least a part of a component after start of decrease in intensity among components of a reflected light pulse generated by irradiation of the first region with the second light pulse and output a second signal according to an amount of the detected light, detect at least a part of a component after start of decrease in intensity among components of a reflected light pulse generated by irradiation of the second region with the first light pulse and output a third signal according to an amount of the detected light, and detect at least a part of a component after start of decrease in intensity among components of a reflected light pulse generated by irradiation of the second region with the second light pulse and output a fourth signal according to an amount of the detected light. The processing circuit may generate a first cerebral blood flow signal indicative of a state of hemoglobin in a cerebral blood flow in the first region on the basis of the first detection signal and the second detection signal, generate a second cerebral blood flow signal indicative of a state of hemoglobin in cerebral blood in the second region on the basis of the third detection signal and the fourth detection signal, and generate the brain activity data on the basis of the first cerebral blood flow signal or the second cerebral blood flow signal. It is possible to estimate a state of brain activity with more accuracy in each region by using two kinds of cerebral blood flow signals each corresponding to light of two wavelengths.

The light emitting device may include a first light source that emits first irradiation light having a first wavelength that is equal to or longer than 650 nm and shorter than 805 nm in air and a second light source that emits second irradiation light having a second wavelength that is longer than 805 nm and equal to or shorter than 950 nm in air. The sensor may be configured to detect reflected light generated by irradiation of the first region with the first irradiation light and output a first detection signal according to an amount of the detected light, detect reflected light generated by irradiation of the first region with the second irradiation light and output a second detection signal according to an amount of the detected light, detect reflected light generated by irradiation of the second region with the first irradiation light and output a third detection signal according to an amount of the detected light, and detect reflected light generated by irradiation of the second region with the second irradiation light and output a fourth detection signal according to an amount of the detected light. The processing circuit may generate a first cerebral blood flow signal indicative of a state of hemoglobin in a cerebral blood flow in the first region on the basis of the first detection signal and the second detection signal, generate a second cerebral blood flow signal indicative of a state of hemoglobin in cerebral blood in the second region on the basis of the third detection signal and the fourth detection signal, and generate the brain activity data on the basis of the first cerebral blood flow signal or the second cerebral blood flow signal. It is possible to estimate a state of brain activity with more accuracy in each region by using two kinds of cerebral blood flow signals each corresponding to light of two wavelengths.

The processing circuit may normally generate the brain activity data on the basis of the detection signal in the first region and may generate the brain activity data on the basis of the detection signal in the second region in a case where a predetermined condition is satisfied. The predetermined condition can be a condition indicating that reliability of a detection signal in the first region is low. For example, the predetermined condition can be a condition that an amount of change in shape of skin detected on the basis of the detection signals or the image signal is larger than a threshold value. The processing circuit can be configured to repeat an operation of selecting the first region as the target region and generating and outputting the brain activity data on the basis of the detection signal in the first region. In this case, the processing circuit may select the second region as the target region instead of the first region and generate the brain activity data on the basis of the detection signal in the second region in a case where the detection signal in the first region and/or the image signal satisfies the predetermined condition.

The present disclosure includes a processing method performed by the biological measurement device and a computer program that defines the processing method. Such a computer program can be stored in a computer-readable non-transitory recording medium.

Embodiments of the present disclosure are more specifically described below with reference to the drawings. Each of the embodiments described below illustrates a general or specific example. Numerical values, shapes, materials, constituent elements, the way in which the constituent elements are disposed and connected, steps, the order of steps, and the like illustrated in the embodiments below are examples and do not limit the present disclosure. Among constituent elements in the embodiments below, constituent elements that are not described in independent claims indicating highest concepts are described as optional constituent elements. The drawings are schematic views and are not necessarily strict illustration. Furthermore, in the drawings, substantially identical or similar constituent elements are given identical reference signs, and repeated description is sometimes omitted or simplified.

### First Embodiment

A biological measurement device according to a first exemplary embodiment of the present disclosure is described. The biological measurement device according to the present embodiment is a non-contact type NIRS device and can measure brain activity of a subject without making contact with subject's skin.

### 1. Configuration

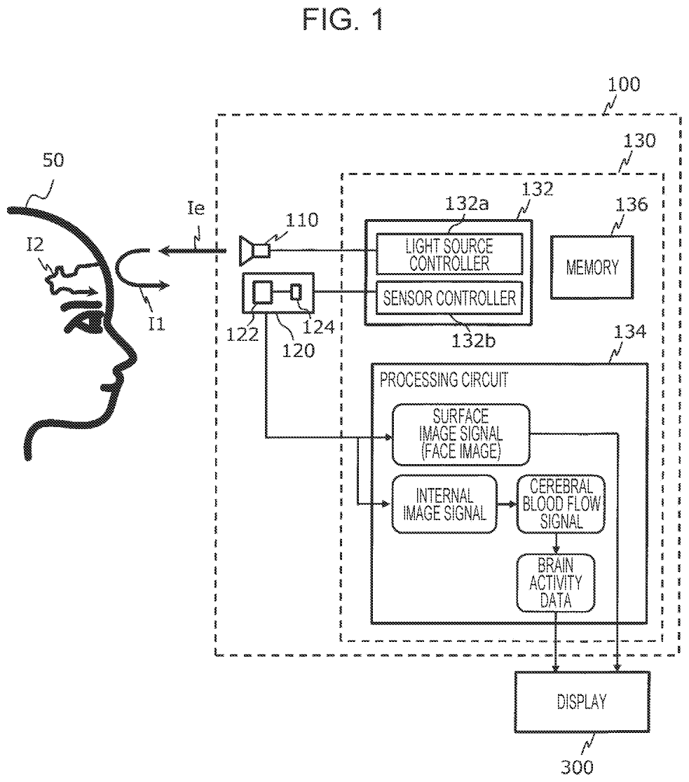

FIG. 1 schematically illustrates a configuration of a biological measurement device 100 according to the first embodiment. This biological measurement device 100 includes a light emitting device 110, an image sensor 120, and a processing device 130. The image sensor 120 is an example of the sensor. The processing device 130 includes a control circuit 132, a signal processing circuit 134, and a storage medium such as a memory 136. The control circuit 132 includes a light source controller 132a and a sensor controller 132b. The light source controller 132a controls the light emitting device 110. The sensor controller 132b controls the image sensor 120. In FIG. 1, a user 50, who is a subject to be measured, and a display 300 are also illustrated. The display 300 may be a constituent element of the measurement device 100 or may be an element outside the measurement device 100.

The light emitting device 110 includes one or more light sources. The image sensor 120 includes photodetection element cells. Each of the photodetection cells includes one or more photoelectric conversion elements 122 and one or more charge accumulation units 124. Although a single photoelectric conversion element 122 and a single charge accumulation unit 124 are illustrated in FIG. 1, photoelectric conversion elements 122 and charge accumulation units 124 can be provided actually.

The light emitting device 110 irradiates a range including a first region and a second region located on an upper side relative to the first region on a forehead of the user 50 with light. The image sensor 120 receives a component of a reflected light pulse generated by irradiation of a light pulse by the light emitting device 110 and outputs, for each photodetection cell, an electric signal according to an amount of received light. The control circuit 132 controls the light emitting device 110 and the image sensor 120. The control circuit 132 repeatedly performs an operation of causing the light emitting device 110 to emit a light pulse toward the forehead of the user 50 and causing the image sensor 120 to detect a specific component of a reflected light pulse reflected back from the forehead. In the present embodiment, the control circuit 132 causes each photodetection cell of the image sensor 120 to detect at least a part of a front end component of the reflected light pulse and at least a part of a rear end component of the reflected light pulse and output an electric signal according to each light amount. The front end component of the reflected light pulse is a component from start of increase in intensity of the reflected light pulse that has reached a light receiving surface of the image sensor 120 to end of the increase. The rear end component of the reflected light pulse is a component from start of decrease in intensity of the reflected light pulse that has reached the light receiving surface of the image sensor 120 to end of the decrease. In the following description, an electric signal indicative of the front end component of the reflected light pulse is sometimes referred to as a “pulse front end signal”, and an electric signal indicative of the rear end component of the reflected light pulse is sometimes referred to as a “pulse rear end signal”. The pulse front end signal is a signal reflecting an intensity of a surface reflected component I1 reflected on a surface of the forehead of the user 50 among components of a light pulse Ie emitted from the light emitting device 110. The pulse rear end signal is a signal reflecting an intensity of an internal scattered component I2 scattered inside the forehead of the user 50 among the components of the light pulse Ie. In the following description, an image signal indicative of a spatial distribution of the intensity of the surface reflected component I1 formed from the pulse front end signal of each pixel is sometimes referred to as a “surface image signal”, and an image signal indicative of a spatial distribution of the intensity of the internal scattered component I2 formed from the pulse rear end signal of each pixel is sometimes referred to as an “internal image signal”. The surface image signal indicates an image including the face of the user 50 and is therefore sometimes referred to as a “face image signal”. The surface image signal may be formed from all components from start of increase in intensity of the reflected light pulse to end of decrease in intensity of the reflected light pulse. The internal image signal is an image signal reflecting a spatial distribution of a cerebral blood flow of the user 50. In the following description, an image signal may be sometimes referred to simply as an “image”.

The processing circuit 134 generates a face image signal indicative of an image including the face of the user 50 on the basis of the pulse front end signal of each pixel. Note that the processing circuit 134 may output the pulse front end signal as the face image signal without particularly processing the pulse front end signal. The processing circuit 134 generates, for each pixel, a cerebral blood flow signal indicative of a state of a cerebral blood flow of the user 50 on the basis of the pulse rear end signal. The cerebral blood flow signal can be a signal indicative of a temporal change of an oxyhemoglobin concentration, a deoxyhemoglobin concentration, or a total hemoglobin concentration, which is a sum of the oxyhemoglobin concentration and the deoxyhemoglobin concentration, in cerebral blood of the user 50. The processing circuit 134 generates brain activity data indicative of a state of brain activity of the user 50 on the basis of the cerebral blood flow signal. The brain activity data can be data indicative of a psychological state or a physical state (e.g., a degree of concentration) of the user 50 estimated from the cerebral blood flow signal. Note that the cerebral blood flow signal or image data indicative of a spatial distribution of the cerebral blood flow signal may be output as the brain activity data.

The processing circuit 134 according to the present embodiment detects movement of skin of the forehead of the user 50 on the basis of a temporal change of the cerebral blood flow signal or the face image signal and selects the first region or the second region as a target region in accordance with an amount of the movement. Then, the processing circuit 134 generates brain activity data on the basis of a detection signal (i.e., the pulse rear end signal) in the selected target region. The generated brain activity data can be, for example, sent to the display 300 together with the face image signal, and an image indicative of a state of brain activity of the user 50 can be displayed.

The following more specifically describes each constituent element according to the present embodiment.

### 1-1. Light Emitting Device 110

The light emitting device 110 is disposed so as to emit light toward a portion to be measured including the head (e.g., forehead) of the user 50. Light that is emitted from the light emitting device 110 and reaches the user 50 is separated into the surface reflected component I1 reflected on a surface of the user 50 and the internal scattered component I2 scattered inside the user 50. The internal scattered component I2 is a component that is reflected or scattered once or multiple-scattered inside the living body. In a case where light is emitted toward a forehead portion of a person as in the present embodiment, the internal scattered component I2 is a component that reaches a portion (e.g., brain) inside the forehead portion that is located 8 mm to 16 mm away from a surface of the forehead portion and returns to the measurement device 100. The surface reflected component I1 includes three components, specifically, a directly reflected component, a diffused reflected component, and a scattered reflected component. The directly reflected component is a reflected component whose incident angle and reflection angle are equal. The diffused reflected component is a component that is reflected by being diffused by irregularities of a surface. The scattered reflected light is a component that is reflected by being scattered by an internal tissue in the vicinity of the surface. The scattered reflected light is a component that is reflected by being scattered inside a surface layer of skin. The surface reflected component I1 can include these three components. The surface reflected component I1 and the internal scattered component I2 change their traveling directions due to reflection or scattering, and a part thereof reaches the image sensor 120. The surface reflected component I1 includes information on a surface of the portion to be measured, for example, information on a blood flow of a face and scalp. The internal scattered component I2 includes information on a user's inside, for example, information on a cerebral blood flow.

In the present embodiment, the surface reflected component I1 and the internal scattered component I2 of reflected light reflected back from the head of the user 50 are detected. The surface reflected component I1 reflects outer appearance of the face or a state of a scalp blood flow of the user 50. It is therefore possible to estimate a change in outer appearance of the face or state of a scalp blood flow of the user 50 by analyzing a temporal change of the surface reflected component I1. Meanwhile, an intensity of the internal scattered component I2 fluctuates reflecting brain activity of the user 50. It is therefore possible to estimate a state of brain activity of the user 50 by analyzing a temporal change of the internal scattered component I2.

The following describes an example of a method for acquiring the internal scattered component I2. The light emitting device 110 repeatedly emits a light pulse plural times at predetermined time intervals or at predetermined timings in accordance with an instruction from the control circuit 132. The light pulse emitted from the light emitting device 110 can be, for example, a rectangular wave whose falling period has a length close to zero. In the present specification, the “falling period” refers to a period from start of decrease in intensity of a light pulse to end of the decrease. Typically, light incident on the head of the user 50 propagates through the head while passing various routes and is emitted from a surface of the head at different timings. Accordingly, a rear end of the internal scattered component I2 of the light pulse has an expanse. In a case where the portion to be measured is a forehead, the expanse of the rear end of the internal scattered component I2 is approximately 4 ns. In consideration of this, the length of the falling period of the light pulse can be, for example, set equal to or less than a half of this value, that is, equal to or less than 2 ns. The falling period may be equal to or less than 1 ns, which is a half of 2 ns. A rising period of the light pulse emitted from the light emitting device 110 can have any length. In the present specification, the “rising period” refers to a period from start of increase in intensity of the light pulse to end of the increase. In detection of the internal scattered component I2 according to the present embodiment, the falling part of the light pulse is used, and the rising part of the light pulse is not used. The rising part of the light pulse is used for detection of the surface reflected component I1.

The light emitting device 110 includes one or more light sources. The light source can include, for example, a laser element such as a laser diode (LD). Light emitted from the laser element can be adjusted to have steep time response characteristics such that a falling part of a light pulse is substantially orthogonal to a time axis. The light emitting device 110 can include a drive circuit that controls a drive current of the LD. The drive circuit can include, for example, an enhancement mode power transistor such as a field-effect transistor (GaNFET) including a gallium nitride (GaN) semiconductor. By using such a drive circuit, falling of the light pulse output from the LD can be made steep.

Light emitted from the light emitting device 110 can have, for example, any wavelength included in a wavelength range equal to or longer than 650 nm and equal to or shorter than 950 nm. This wavelength range is included in a wavelength range from red to near-infrared rays. This wavelength range is called a “biological window” and has such a property that light is relatively hard to be absorbed by moisture in a living body and skin. In a case where a living body is used as a detection target, detection sensitivity can be increased by using light in this wavelength range. Note that in the present specification, a term “light” is used not only for visible light, but also for an infrared ray. In a case where a change in cerebral blood flow of a person is detected as in the present embodiment, used light is considered to be mainly absorbed by oxyhemoglobin (HbO₂) and deoxyhemoglobin (Hb). The oxyhemoglobin and the deoxyhemoglobin are different in wavelength dependence of light absorption. In general, when a blood flow changes in accordance with brain activity, a concentration of the oxyhemoglobin and a concentration of the deoxyhemoglobin change. A degree of absorption of light also changes in accordance with this change. Accordingly, when the blood flow changes, an amount of detected light also changes temporally. By detecting the change in amount of light, a state of brain activity can be estimated.

The light emitting device 110 may emit light of a single wavelength included in the wavelength range or may emit light of two or more wavelengths included in the wavelength range. The light of the wavelengths may be emitted from light sources.

In general, absorption characteristics and scattering characteristics of a living body tissue vary depending on a wavelength. Therefore, more detailed component analysis of a target to be measured can be conducted by detecting wavelength dependence of an optical signal based on the internal scattered component I2. For example, in a living body tissue, in a case where the wavelength is equal to or longer than 650 nm and is shorter than 805 nm, a coefficient of light absorption by deoxyhemoglobin is higher than a coefficient of light absorption by oxyhemoglobin. On the other hand, in a case where the wavelength is longer than 805 nm and is equal to or shorter than 950 nm, the coefficient of light absorption by oxyhemoglobin is higher than the coefficient of light absorption by deoxyhemoglobin.

Therefore, the light emitting device 110 may be configured to emit light of a wavelength equal to or longer than 650 nm and shorter than 805 nm (e.g., approximately 750 nm) and light of a wavelength longer than 805 nm and equal to or shorter than 950 nm (e.g., approximately 850 nm). In this case, a light intensity of the internal scattered component I2 generated, for example, by light of a wavelength of approximately 750 nm and a light intensity of the internal scattered component I2 generated, for example, by light of a wavelength of approximately 850 nm are measured. The light emitting device 110 may include a light source that emits light of a wavelength equal to or longer than 650 nm and shorter than 805 nm and a light source that emits light of a wavelength longer than 805 nm and equal to or shorter than 950 nm. The processing circuit 134 can find change amounts of concentrations of oxyhemoglobin (HbO₂) and deoxyhemoglobin (Hb) in the blood from initial values by solving predetermined simultaneous equations on the basis of signal values of the light intensities input for each pixel.

The measurement device 100 according to the present embodiment can measure a cerebral blood flow amount of the user 50 in a non-contact manner. For this purpose, the light emitting device 110 designed in consideration of influence on a retina can be used. For example, the light emitting device 110 that satisfies Class 1 of the laser safety standards adopted in various countries can be used. In a case where Class 1 is satisfied, the user 50 is irradiated with light having such low illuminance that an accessible emission limit (AEL) is lower than 1 mW. Note that the light emitting device 110 itself need not satisfy Class 1. For example, Class 1 of the laser safety standards may be satisfied by disposing a diffusion plate or an ND filter in front of the light emitting device 110 and thereby diffusing or attenuating light.

Conventionally, a streak camera is used to detect information such as absorption coefficients or scattering coefficients in different places in a depth direction inside a living body while distinguishing them. For example, Japanese Unexamined Patent Application Publication No. 4-189349 discloses an example of such a streak camera. In such a streak camera, an ultrashort light pulse having a femtosecond or picosecond pulse width is used for measurement at desired spatial resolution.

On the other hand, the measurement device 100 according to the present embodiment can detect the surface reflected component I1 and the internal scattered component I2 while distinguishing the surface reflected component I1 and the internal scattered component I2. Therefore, the light pulse emitted by the light emitting device 110 need not be an ultrashort light pulse, and can have any pulse width.

In a case where the head of a person is irradiated with light to measure a cerebral blood flow, a light amount of the internal scattered component I2 can be a very small value that is approximately one-several thousandth to one-several ten thousandth of a light amount of the surface reflected component I1. Furthermore, an amount of light that can be emitted is extremely small in consideration of the laser safety standards. It is therefore very difficult to detect the internal scattered component I2. Even in this case, in a case where the light emitting device 110 emits a light pulse having a relatively large pulse width, an integrated amount of the internal scattered component I2 involving a time delay can be increased. This can increase a detected light amount and improve a signal-to-noise (SN) ratio.

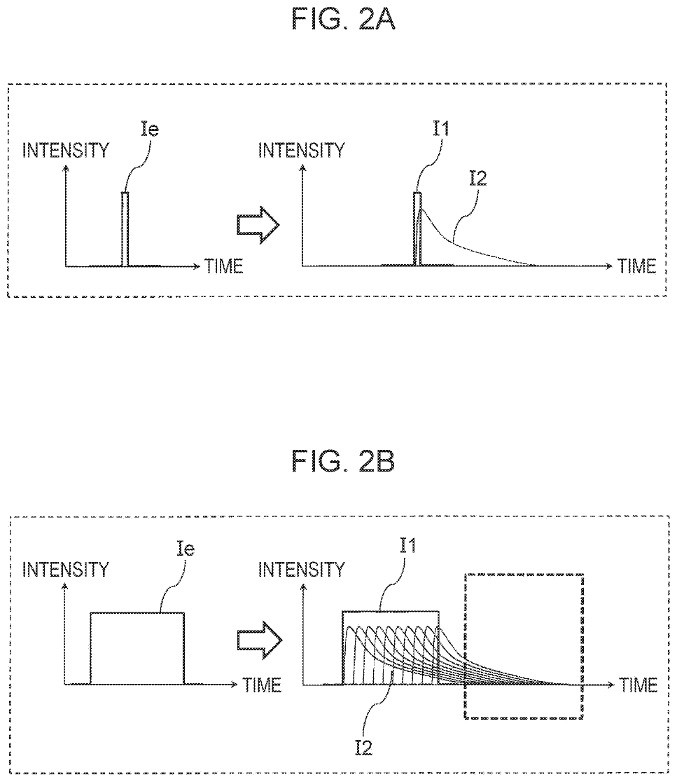

FIGS. 2A and 2B illustrate examples of temporal changes of the intensity of the emitted light pulse Ie and the intensities of the surface reflected component I1 and the internal scattered component I2 in the reflected light pulse. FIG. 2A illustrates an example of waveforms obtained in a case where the emitted light pulse Ie has an impulse waveform. FIG. 2B illustrates an example of waveforms obtained in a case where the emitted light pulse Ie has a rectangular waveform. Although the internal scattered component I2 is actually weak, the intensity of the internal scattered component I2 is emphasized in FIGS. 2A and 2B.

As illustrated in FIG. 2A, in a case where the emitted light pulse Ie has an impulse waveform, the surface reflected component I1 has an impulse waveform similar to the light pulse Ie, and the internal scattered component I2 has an impulse response waveform delayed relative to the surface reflected component I1. This is because the internal scattered component I2 corresponds to a combination of light beams that have passed various routes inside the skin.

As illustrated in FIG. 2B, in a case where the light pulse Ie has a rectangular waveform, the surface reflected component I1 has a rectangular waveform similar to the light pulse Ie, and the internal scattered component I2 has a waveform in which a large number of impulse response waveforms are superimposed. The inventors of the present invention confirmed that the light amount of the internal scattered component I2 detected by the image sensor 120 can be amplified by superimposition of a large number of impulse response waveforms as compared with a case where the light pulse Ie has an impulse waveform. The internal scattered component I2 can be effectively detected by starting opening of an electronic shutter at or after a timing of start of falling of the reflected light pulse. The broken-line frame on the right side of FIG. 2B illustrates an example of a shutter opening period for which the electronic shutter of the image sensor 120 is opened. This shutter opening period is also referred to as an “exposure period”. In a case where a pulse width of the rectangular pulse is 1 ns to 10 ns order, the light emitting device 110 can be driven at a relatively low voltage, and a reduction in size and cost of the measurement device 100 can be achieved. The internal scattered component I2 can be effectively detected by starting exposure at or after a timing of falling of the surface reflected component I1 reaching the image sensor 120.

The light emitting device 110 can include, for example, a light-emitting element using a general-purpose semiconductor laser. In a case where the general-purpose semiconductor laser is driven at a low voltage, setting the pulse width too short makes following of driving of ON and OFF of light hard. Accordingly, an emitted light waveform varies from one pulse emission to another. As a result, an unstable behavior is likely to be exhibited, and variations in measurement result are likely to be caused. The light emitting device 110 can be, for example, controlled to emit a light pulse having a pulse width of 3 ns or more to obtain a stable waveform by using a general-purpose semiconductor laser. Alternatively, the light emitting device 110 may emit a light pulse having a pulse width of 5 ns or more or a pulse width of 10 ns or more to further stabilize the waveform. On the other hand, setting the pulse width too large increases a light flow to the charge accumulation unit 124 in a shutter OFF state, that is, increases parasitic light sensitivity (PLS), leading to a risk of a measurement error. In view of this, the light emitting device 110 can be, for example, controlled to generate a light pulse having a pulse width of 50 ns or less. Alternatively, the light emitting device 110 may emit a light pulse having a pulse width of 30 ns or less or a pulse width of 20 ns or less.

The biological measurement device 100 according to the present embodiment can reduce the surface reflected component I1 while temporally separating the surface reflected component I1 from the internal scattered component I2. Therefore, for example, a pattern having a uniform intensity distribution within an irradiation region can be selected as an irradiation pattern of the light emitting device 110. In this case, the user 50 can be irradiated with light of illuminance that is equal spatially, and a detection signal of an intensity that falls within a dynamic range can be acquired in any pixel of the image sensor 120. The irradiation pattern having a uniform intensity distribution may be formed by diffusing light emitted from the light emitting device 110 by using a diffusion plate.

### 1-2. Image Sensor 120

The image sensor 120 can be, for example, any imaging element such as a CCD image sensor or a CMOS image sensor. The image sensor 120 includes photodetection cells that are two-dimensionally disposed on the light receiving surface. Each of the photodetection cells can include, for example, a photoelectric conversion element such as a photodiode and one or more charge accumulation units. The photoelectric conversion element generates a signal charge according to an amount of received light by photoelectric conversion. The charge accumulation unit accumulates the signal charge generated by the photoelectric conversion element. The image sensor 120 can acquire two-dimensional information of a user at one time. In the following description, the photodetection cells are sometimes referred to as “pixels”.

The image sensor 120 according to the present embodiment includes an electronic shutter. The electronic shutter is a circuit that controls a timing of exposure. The electronic shutter controls a period of one signal accumulation for which received light is converted into an effective electric signal and is accumulated and a period for which the signal accumulation is stopped. The signal accumulation period is referred to as an “exposure period”. A period from end of one exposure period to start of a next exposure period is referred to as a “non-exposure period”. Hereinafter, a state where exposure is being performed is sometimes referred to as “OPEN, and a state where the exposure is stopped is sometimes referred to as “CLOSED”.

The image sensor 120 can adjust the exposure period and the non-exposure period within a subnanosecond range, for example, a range from 30 ps to 1 ns by the electronic shutter. The exposure period and the non-exposure period can be, for example, set to a value equal to or larger than 1 ns and equal to or smaller than 30 ns.

In a case where information such as a cerebral blood flow is detected by irradiating a forehead of a person with light, a rate of attenuation of light inside the living body is very large. For example, emitted light can attenuate to approximately one-millionth of incident light. Accordingly, a light amount sufficient to detect an internal scattered component cannot be sometimes obtained by irradiation of one pulse. Especially in a case of irradiation of Class 1 of the laser safety standards, a light amount is weak. In this case, the control circuit 132 causes the light emitting device 110 to emit a light pulse plural times, and the photodetection cells of the image sensor 120 are exposed to light plural times in synchronization with this. By thus integrating signals plural times, sensitivity can be improved.

The following describes an example in which each pixel of the image sensor 120 includes a photoelectric conversion element such as a photodiode and charge accumulation units. The charge accumulation units in each pixel can include a charge accumulation unit that accumulates a signal charge generated by a surface reflected component of a light pulse and a charge accumulation unit that accumulates a signal charge generated by an internal scattered component of the light pulse. The control circuit 132 causes the image sensor 120 to detect a component before start of falling in a reflected light pulse reflected back from a forehead of a user and thereby detect a surface reflected component. The control circuit 132 causes the image sensor 120 to detect a component after start of falling in a light pulse reflected back from a portion to be measured of the user and thereby detect an internal scattered component.

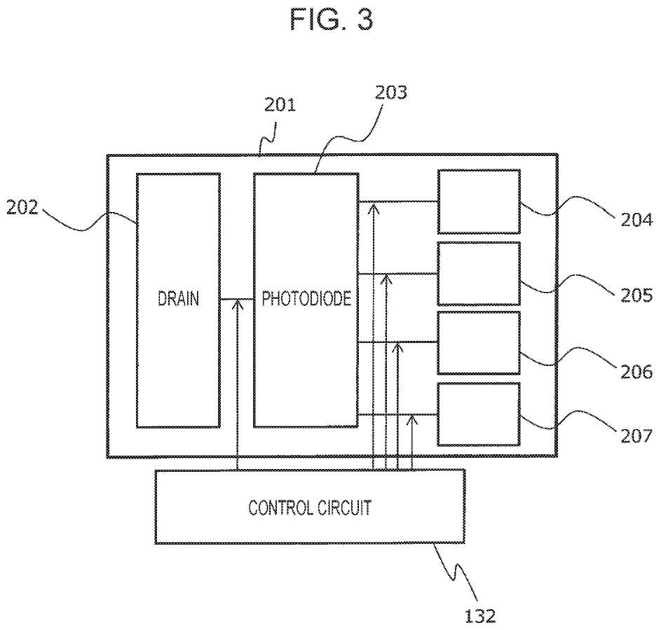

FIG. 3 illustrates an outline configuration of one pixel 201 of the image sensor 120. Note that FIG. 3 schematically illustrates the configuration of one pixel 201, and an actual structure is not necessarily reflected in FIG. 3. The pixel 201 in this example includes a photodiode 203 that performs photoelectric conversion, a first floating diffusion (FD) layer 204, a second floating diffusion layer 205, a third floating diffusion layer 206, and a fourth floating diffusion layer 207, which are charge accumulation units, and a drain 202 to which a signal charge is discharged. In the example illustrated in FIG. 3, the light emitting device 110 emits light pulses of two kinds of wavelengths.

A photon entering each pixel due to one emission of a light pulse is converted into a signal electron, which is a signal charge, by the photodiode 203. The signal electron is discharged to the drain 202 or is sorted into any one of the first to fourth floating diffusion layers 204 to 207 in accordance with a control signal input from the control circuit 132 to the image sensor 120.

Emission of a light pulse from the light emitting device 110, accumulation of a signal charge in any of the first floating diffusion layer 204, the second floating diffusion layer 205, the third floating diffusion layer 206, and the fourth floating diffusion layer 207, and discharge of a signal charge to the drain 202 are repeatedly performed in this order. This operation is repeated at a high rate, and can be, for example, repeated several tens of thousands to several hundreds of millions of times within a period of one frame of a moving image. The period of one frame can be, for example, approximately 1/30 seconds. The pixel 201 finally generate and output, for each frame, four image signals based on the signal charges accumulated in the first to fourth floating diffusion layers 204 to 207.

The control circuit 132 according to the present embodiment causes the light emitting device 110 to emit a first light pulse having a first wavelength λ1 and a second light pulse having a second wavelength λ2. An internal state of a portion to be measured can be analyzed by selecting two wavelengths that are different in rate of absorption in an internal tissue of the portion to be measured as the wavelengths λ1 and λ2. For example, a wavelength equal to or longer than 650 nm and shorter than 805 nm can be selected as the wavelength λ1, and a wavelength longer than 805 nm and equal to or shorter than 950 nm can be selected as the wavelength λ2. This makes it possible to efficiently detect a change in oxyhemoglobin concentration and a change in deoxyhemoglobin concentration in the blood of the user 50, as described later.

The control circuit 132 performs, for example, the following operation. The control circuit 132 causes the light emitting device 110 to emit a light pulse of the wavelength λ1, and causes the first floating diffusion layer 204 to accumulate a signal charge during a period where an internal scattered component of the light pulse is incident on the photodiode 203. The control circuit 132 causes the light emitting device 110 to emit the light pulse of the wavelength λ, and causes the second floating diffusion layer 205 to accumulate a signal charge during a period where a surface reflected component of the light pulse is incident on the photodiode 203. Furthermore, the control circuit 132 causes the light emitting device 110 to emit a light pulse of the wavelength λ2, and causes the third floating diffusion layer 206 to accumulate a signal charge during a period where an internal scattered component of the light pulse is incident on the photodiode 203. The control circuit 132 causes the light emitting device 110 to emit a light pulse of the wavelength λ2, and causes the fourth floating diffusion layer 207 to accumulate a signal charge during a period where a surface reflected component of the light pulse is incident on the photodiode 203. The above operation can be repeated plural times. By such an operation, an image showing a two-dimensional distribution of the surface reflected component and an image showing a two-dimensional distribution of the internal scattered component can be acquired for both of the wavelength λ1 and the wavelength λ2.

To estimate light amounts of disturbance light and environment light, a period where a signal charge is accumulated in another floating diffusion layer (not illustrated) in a state where the light emitting device 110 is off may be provided. A signal excluding disturbance light and environment light components can be obtained by subtracting a signal charge amount of the other floating diffusion layer from signal charge amounts of the first to fourth first floating diffusion layers 204 to 207.

Note that although the number of charge accumulation units of each pixel is four in the present embodiment, the number of charge accumulation units of each pixel may be set to any number of 1 or more depending on a purpose. For example, in a case where a surface reflected component and an internal scattered component are detected by using one kind of wavelength, the number of charge accumulation units may be two. In a case where one kind of wavelength is used and a surface reflected component is not detected, the number of charge accumulation units of each pixel may be one. In a case where an internal scattered component is detected by using two kinds of wavelengths, the number of charge accumulation units of each pixel may be two. Even in a case where two or more kinds of wavelengths are used, the number of charge accumulation units may be one, as long as imaging using one wavelength and imaging using another wavelength are performed in different frames. Similarly, even in a case where both of a surface reflected component and an internal scattered component are detected, the number of charge accumulation units may be one, as long as the surface reflected component and the internal scattered component are detected in different frames.

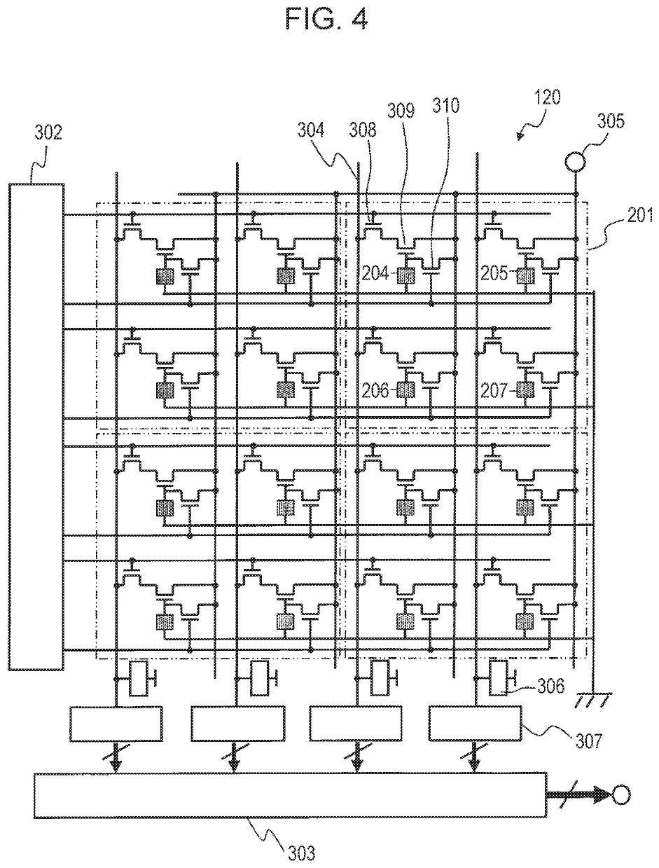

Next, an example of the configuration of the image sensor 120 is described in more detail with reference to FIG. 4.

FIG. 4 is a diagram illustrating an example of the configuration of the image sensor 120. In FIG. 4, a region surrounded by the line with alternate long and two short dashes corresponds to a single pixel 201. The pixel 201 includes a single photodiode. Although four pixels arranged in two rows and two columns are illustrated in FIG. 4, a larger number of pixels can be disposed actually. The pixel 201 includes the first to fourth floating diffusion layers 204 to 207. Signals accumulated in the first to fourth floating diffusion layers 204 to 207 are handled as if these signals are signals of four pixels of a general CMOS image sensor, and are output from the image sensor 120.

Each pixel 201 has four signal detection circuits. Each signal detection circuit includes a source follower transistor 309, a row selection transistor 308, and a reset transistor 310. In this example, the reset transistor 310 corresponds to the drain 202 illustrated in FIG. 3, and a pulse input to a gate of the reset transistor 310 corresponds to the drain discharge pulse. Each transistor is, for example, a field-effect transistor provided on a semiconductor substrate but is not limited to this. As illustrated in FIG. 4, one of an input terminal and an output terminal of the source follower transistor 309 is connected to one of an input terminal and an output terminal of the row selection transistor 308. The one of the input terminal and the output terminal of the source follower transistor 309 is typically a source. The one of the input terminal and the output terminal of the row selection transistor 308 is typically a drain. A gate of the source follower transistor 309, which is a control terminal, is connected to the photodiode 203. A signal charge, which is a hole or an electron, generated by the photodiode 203 is accumulated in a floating diffusion layer, which is a charge accumulation unit, provided between the photodiode 203 and the source follower transistor 309.

The first to fourth floating diffusion layers 204 to 207 are connected to the photodiode 203 (not illustrated in FIG. 4). One or more switches can be provided between the photodiode 203 and each of the first to fourth floating diffusion layers 204 to 207. The switch switches a conduction state between the photodiode 203 and each of the first to fourth floating diffusion layers 204 to 207 in accordance with a signal accumulation pulse from the control circuit 132. In this way, start and stop of accumulation of a signal charge in each of the first to fourth floating diffusion layers 204 to 207 are controlled. The electronic shutter according to the present embodiment has a mechanism for such exposure control.

Signal charges accumulated in the first to fourth floating diffusion layers 204 to 207 are read out when a gate of the row selection transistor 308 is turned on by a row selection circuit 302. At this time, a current flowing from a source follower power source 305 to the source follower transistor 309 and a source follower load 306 is amplified in accordance with the signal charges of the first to fourth floating diffusion layers 204 to 207. An analog signal based on this current read out from a vertical signal line 304 is converted into a digital signal data by an analog-digital (AD) conversion circuit 307 connected to each column. This digital signal data is read out for each column by a column selection circuit 303 and is output from the image sensor 120. The row selection circuit 302 and the column selection circuit 303 perform readout in one row and then perform readout in a next row. Thereafter, similarly, information on signal charges of floating diffusion layers in all rows is read out. The control circuit 132 turns the gate of the reset transistor 310 on after all signal charges are read out, and thereby resets all floating diffusion layers. This completes imaging of one frame. Thereafter, similarly, high-rate imaging of a frame is repeated, and thereby imaging of a series of frames by the image sensor 120 is completed.

Although an example in which a CMOS-type image sensor 120 is used has been described in the present embodiment, the image sensor 120 may be another kind of imaging element. For example, the image sensor 120 may be a CCD type, may be a single photon counting type element, or may be an amplification type image sensor such as an EMCCD or an ICCD. Furthermore, sensors each including a single photoelectric conversion element may be used instead of the image sensor 120 having photodetection cells that are two-dimensionally arranged. Even in a case where single-pixel sensors are two-dimensionally arranged, two-dimensional data of a portion to be measured can be generated.

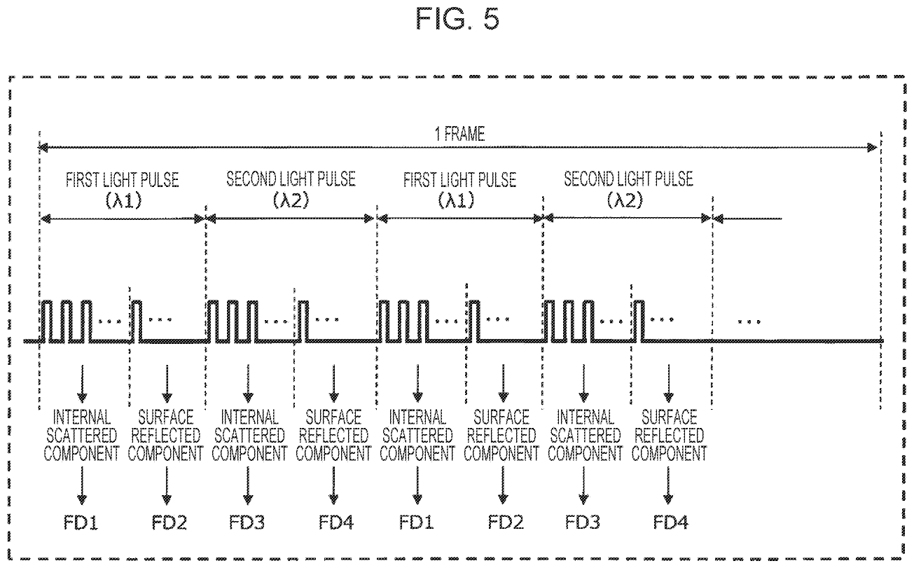

FIG. 5 schematically illustrates an example of an operation performed in one frame. In the example illustrated in FIG. 5, a period for which the first light pulse of the wavelength λ1 is repeatedly emitted and a period for which the second light pulse of the wavelength λ2 is repeatedly emitted alternate within a single frame. The period for which the first light pulse is repeatedly emitted and the period for which the second light pulse is repeatedly emitted each include a period for which a signal charge of an internal scattered component is accumulated and a period for which a signal charge of a surface reflected component is accumulated. The internal scattered component of the light pulse of the wavelength λ1 is accumulated in the first floating diffusion layer 204 (FD1). The surface reflected component of the light pulse of the wavelength λ1 is accumulated in the second floating diffusion layer 205 (FD2). The internal scattered component of the light pulse of the wavelength λ2 is accumulated in the third floating diffusion layer 206 (FD3). The surface reflected component of the light pulse of the wavelength λ2 is accumulated in the fourth floating diffusion layer 207 (FD4). In this example, the control circuit 132 repeats the following operations (i) to (iv) plural times within a one-frame period.
(i) An operation of causing the light emitting device 110 to emit the light pulse of the wavelength λ1 and causing the first floating diffusion layer 204 of each pixel to accumulate the internal scattered component of the light pulse of the wavelength λ1 is repeated a predetermined number of times.
(ii) An operation of causing the light emitting device 110 to emit the light pulse of the wavelength λ1 and causing the second floating diffusion layer 205 of each pixel to accumulate the surface reflected component of the light pulse of the wavelength λ1 is repeated plural times.
(iii) An operation of causing the light emitting device 110 to emit the light pulse of the wavelength λ2 and causing the third floating diffusion layer 206 of each pixel to accumulate the internal scattered component of the light pulse of the wavelength λ2 is repeated a predetermined number of times.
(iv) An operation of causing the light emitting device 110 to emit the light pulse of the wavelength λ2 and causing the fourth floating diffusion layer 207 of each pixel to accumulate the surface reflected component of the light pulse of the wavelength λ2 is repeated plural times.

By such operations, a temporal difference between timings of acquisition of detection signals using two kinds of wavelengths can be reduced, and imaging using the first light pulse and imaging using the second light pulse can be performed almost simultaneously.

In the present embodiment, the image sensor 120 detects a surface reflected component and an internal scattered component for each of the first light pulse and the second light pulse and generate an image signal indicative of an intensity distribution of each component. A cerebral blood flow signal of the user 50 can be generated for each pixel or each pixel group on the basis of an image signal indicative of an intensity distribution of the internal scattered component of each of the first light pulse and the second light pulse. On the other hand, an image signal indicative of an intensity distribution of the surface reflected component of each of the first light pulse and the second light pulse indicates a face image of the user 50. On the basis of a temporal change of the face image signal, the processing circuit 134 can decide a region of the forehead of the user 50 and generate brain activity data by using a detection signal in the decided region.

Note that the light emitting device 110 may emit light of one kind of wavelength. Even in this case, an approximate state of brain activity can be estimated.

### 1-3. Processing Device 130

The processing device 130 includes the control circuit 132, the processing circuit 134, and the memory 136.

The control circuit 132 controls the above operations of the light emitting device 110 and the image sensor 120. Specifically, the control circuit 132 adjusts a time difference between an emission timing of a light pulse of the light emitting device 110 and a shutter timing of the image sensor 120. Hereinafter, the time difference is sometimes referred to as a “phase difference”. The “emission timing” of the light emitting device 110 refers to a timing of start of rising of a light pulse emitted from the light emitting device 110. The “shutter timing” refers to a timing of start of exposure.

The control circuit 132 can be, for example, a processor such as a central processing unit (CPU) or an integrated circuit such as a microcontroller including a processor. The control circuit 132 adjusts the emission timing and the shutter timing, for example, by execution of a computer program recorded in the memory 136 by the processor.

The processing circuit 134 is a circuit that processes a signal output from the image sensor 120. The processing circuit 134 performs arithmetic processing such as image processing. The processing circuit 134 can be, for example, realized by a digital signal processor (DSP), a programmable logic device (PLD) such as a field programmable gate array (FPGA), a central processing unit (CPU), or a graphics processing unit (GPU). The processing circuit 134 performs processing that will be described later by execution of a computer program stored in the memory 136 by a processor.

The memory 136 is a recording medium such as a ROM or a RAM in which computer programs executed by the control circuit 132 and the processing circuit 134 and various kinds of data generated by the control circuit 132 and the processing circuit 134 are recorded.

The control circuit 132 and the processing circuit 134 may be a single unified circuit or may be separate individual circuits. The control circuit 132 and the processing circuit 134 may each include circuits. At least one function of the processing circuit 134 may be a constituent element of an external device such as a server provided separately from the light emitting device 110 and the image sensor 120. In this case, the external device transmits and receives data to and from the measurement device including the light emitting device 110, the image sensor 120, and the control circuit 132 through wireless communication or wired communication.

The processing circuit 134 can generate an image signal reflecting the surface reflected component I1 and a cerebral blood flow signal reflecting the internal scattered component I2 on the basis of a pulse front end signal and a pulse rear end signal output from the image sensor 120. The processing circuit 134 can generate a face image signal of the user 50 on the basis of a pulse front end signal of each pixel output for each frame from the image sensor 120. The processing circuit 134 can generate moving image data indicative of temporal changes of concentrations of oxyhemoglobin, deoxyhemoglobin, and total hemoglobin in the blood inside the portion to be measured on the basis of a pulse rear end signal of each pixel output for each frame from the image sensor 120. The processing circuit 134 can also generate brain activity data indicative of a psychological state or a physical state (e.g., a degree of concentration) of the user 50 on the basis of information on these concentrations. Note that the processing circuit 134 may generate not only such data, but also other data. For example, the processing circuit 134 may generate brain activity data including information such as a blood oxygen saturation level.

The processing circuit 134 may estimate an offset component resulting from disturbance light included in a signal output from the image sensor 120 and remove the offset component. The offset component is a signal component resulting from disturbance light such as solar light or fluorescent light. The offset component resulting from environment light or disturbance light is estimated by causing the image sensor 120 to detect a signal in a state where no light is emitted by turning driving of the light emitting device 110 off.

### 1-4. Other Remarks

The measurement device 100 may include an imaging optical system that forms a two-dimensional image of the user 50 on the light receiving surface of the image sensor 120. An optical axis of the imaging optical system is substantially orthogonal to the light receiving surface of the image sensor 120. The imaging optical system may include a zoom lens. When a position of the zoom lens changes, a magnification of the two-dimensional image of the user 50 changes, and resolution of the two-dimensional image on the image sensor 120 changes. Therefore, a desired measurement region can be enlarged and observed in detail even in a case where a distance to the user 50 is long.

The measurement device 100 may include, between the user 50 and the image sensor 120, a bandpass filter that allows light of a wavelength band emitted from the light emitting device 110 or light in the vicinity of the wavelength band to pass therethrough. This can reduce influence of a disturbance component such as environment light. The bandpass filter can be, for example, a multi-layer filter or an absorption filter. The bandpass filter may have, for example, a bandwidth range of approximately 20 nm to 100 nm in consideration of a band shift resulting from a change in temperature of the light emitting device 110 and oblique incidence on the filter.

The measurement device 100 may include a polarization plate between the light emitting device 110 and the user 50 and between the image sensor 120 and the user 50. In this case, a polarization direction of the polarization plate disposed on the light emitting device 110 side and a polarization direction of the polarization plate disposed on the image sensor 120 side can have a relationship of crossed Nicols. This can prevent a specular reflection component of a surface reflected component of the user 50, that is, a component whose incident angle and reflection angle are identical from reaching the image sensor 120. That is, it is possible to reduce a light amount of the surface reflected component reaching the image sensor 120.

### 2. Operation

Next, an example of an operation according to the present embodiment is described.

### 2-1. Example of Operation of Detecting Surface Reflected Component and Internal Scattered Component

The measurement device 100 according to the present embodiment can detect the surface reflected component I1 and the internal scattered component I2 in a reflected light pulse from a portion to be measured while distinguishing the surface reflected component I1 and the internal scattered component I2. In a case where the portion to be measured is a forehead, a signal intensity of the internal scattered component I2 to be detected is very small. This is because light of a very small light amount that satisfies the laser safety standards is emitted as described above and scattering and absorption of light by a scalp, a cerebral fluid, a skull bone, gray matter, white matter, and blood are large. Furthermore, a change in signal intensity caused by a change in blood flow amount or component in a blood flow during brain activity is one-several tenth of a signal intensity before the change and is very small. Therefore, in a case where the internal scattered component I2 is detected, the surface reflected component I1, which is several thousands to several tens of thousands of times larger than the internal scattered component to be detected, is removed to a maximum extent.

As described above, when the light emitting device 110 irradiates the user 50 with a light pulse, the surface reflected component I1 and the internal scattered component I2 are generated. Part of the surface reflected component I1 and part of the internal scattered component I2 reach the image sensor 120. The internal scattered component I2 passes the inside of the user 50 after emission from the light emitting device 110 until the internal scattered component I2 reaches the image sensor 120. Accordingly, an optical path length of the internal scattered component I2 is longer than an optical path length of the surface reflected component IL Therefore, a timing at which the internal scattered component I2 reaches the image sensor 120 is later than a timing at which the surface reflected component I1 reaches the image sensor 120 on average.

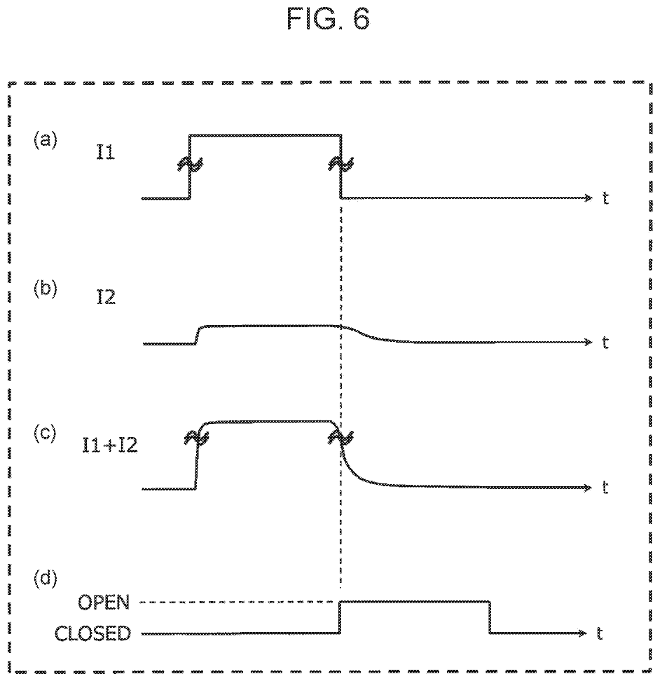

FIG. 6 schematically illustrates a waveform of a light intensity of a reflected light pulse reflected back from the portion to be measured of the user 50 in a case where a rectangular-wave light pulse is emitted from the light emitting device 110. Each horizontal axis represents a time (t). The vertical axis represents an intensity in (a) to (c) of FIG. 6, and represents an OPEN or CLOSED state of the electronic shutter in (d) of FIG. 6. (a) of FIG. 6 illustrates the surface reflected component IL (b) of FIG. 6 illustrates the internal scattered component I2. (c) of FIG. 6 illustrates a sum of the surface reflected component I1 and the internal scattered component I2. As illustrated in (a) of FIG. 6, the surface reflected component I1 maintains an almost rectangular waveform. On the other hand, the internal scattered component I2 is a combination of light beams of various optical path lengths. Accordingly, as illustrated in (b) of FIG. 6, the internal scattered component I2 exhibits such a characteristic that a rear end of the light pulse has a long tail-like shape. In other words, a falling period of the internal scattered component I2 is longer than a falling period of the surface reflected component I1. To extract the internal scattered component I2 from the optical signal illustrated in (c) of FIG. 6 at a high percentage, exposure of the electronic shutter is started at or after a timing at which the rear end of the surface reflected component I1 reaches the image sensor 120, as illustrated in (d) of FIG. 6. In other words, exposure is started at or after a time of falling of the waveform of the surface reflected component I1. This shutter timing is adjusted by the control circuit 132.

In a case where the portion to be measured is not flat, a timing of arrival of light differs from one pixel to another of the image sensor 120. In this case, the shutter timing illustrated in (d) of FIG. 6 may be individually decided for each pixel. For example, assume that a direction orthogonal to the light receiving surface of the image sensor 120 is a z direction. The control circuit 132 may acquire data indicative of a two-dimensional distribution of a z coordinate on a surface of the portion to be measured and vary the shutter timing from one pixel to another on the basis of this data. This makes it possible to decide an optimal shutter timing at each position even in a case where the surface of the portion to be measured is curved. The data indicative of the two-dimensional distribution of the z coordinate on the surface of the portion to be measured is, for example, acquired by a Time-of-Flight (TOF) technique. In the TOF technique, a period it takes for light emitted by the light emitting device 110 to reach each pixel after being reflected by the portion to be measured is measured. A distance between each pixel and the portion to be measured can be estimated on the basis of a difference between a phase of reflected light detected by the pixel and a phase of the light emitted by the light emitting device 110. In this way, the data indicative of the two-dimensional distribution of the z coordinate on the surface of the portion to be measured can be acquired. The data indicative of the two-dimensional distribution can be acquired before measurement.

In the example illustrated in (a) of FIG. 6, the rear end of the surface reflected component I1 falls vertically. In other words, a period from start to end of falling of the surface reflected component I1 is zero. However, actually, the rear end of the surface reflected component I1 does not fall vertically in some cases. For example, in a case where falling of a waveform of a light pulse emitted from the light emitting device 110 is not completely vertical, in a case where the surface of the portion to be measured has minute irregularities, or in a case where scattering occurs in a surface layer of skin, the rear end of the surface reflected component I1 does not vertically fall. Furthermore, since the user 50 is a non-transparent object, a light amount of the surface reflected component I1 is far larger than a light amount of the internal scattered component I2. Therefore, even in a case where the rear end of the surface reflected component I1 is slightly deviated from a point of vertical falling, there is a possibility that the internal scattered component I2 is buried. Furthermore, a time delay resulting from movement of electrons may occur during a readout period of the electronic shutter. For the above reasons, ideal binary readout such as the one illustrated in (d) of FIG. 6 cannot be sometimes realized. In this case, the control circuit 132 may make a timing of shutter start of the electronic shutter slightly, for example, by approximately 0.5 ns to 5 ns later than a timing immediately after falling of the surface reflected component I1. The control circuit 132 may adjust the emission timing of the light emitting device 110 instead of adjusting the shutter timing of the electronic shutter. In other words, the control circuit 132 may adjust a time difference between the shutter timing of the electronic shutter and the emission timing of the light emitting device 110. In a case where a change in blood flow amount or component in blood in the portion to be measured is measured in a non-contact manner, delaying the shutter timing too much further reduces the internal scattered component I2, which is small from the start. Therefore, the shutter timing may be kept in the vicinity of the rear end of the surface reflected component I1. As described above, a time delay caused by scattering inside the portion to be measured is approximately 4 ns. In this case, a maximum amount of delay of the shutter timing can be approximately 4 ns.

As in the example illustrated in FIG. 5A, light pulses may be emitted from the light emitting device 110, and signals may be accumulated by performing exposure for each of the light pulses at shutter timings whose time differences are equal. This amplifies a detected light amount of the internal scattered component I2.

The offset component may be estimated by performing imaging for the same exposure period in a state where no light is emitted by the light emitting device 110 instead of or in addition to disposing a bandpass filter between the user and the image sensor 120. The estimated offset component is removed by subtraction from a signal detected by each pixel of the image sensor 120. This makes it possible to remove a dark current component generated on the image sensor 120.

The internal scattered component I2 includes information on the inside of the user 50 such as cerebral blood flow information. An amount of light absorbed by blood changes in accordance with a temporal change in cerebral blood flow amount of the user 50. As a result, an amount of light detected by the image sensor 120 increases or decreases accordingly. It is therefore possible to estimate a state of brain activity from the change in cerebral blood flow amount of the user 50 by monitoring the internal scattered component I2.

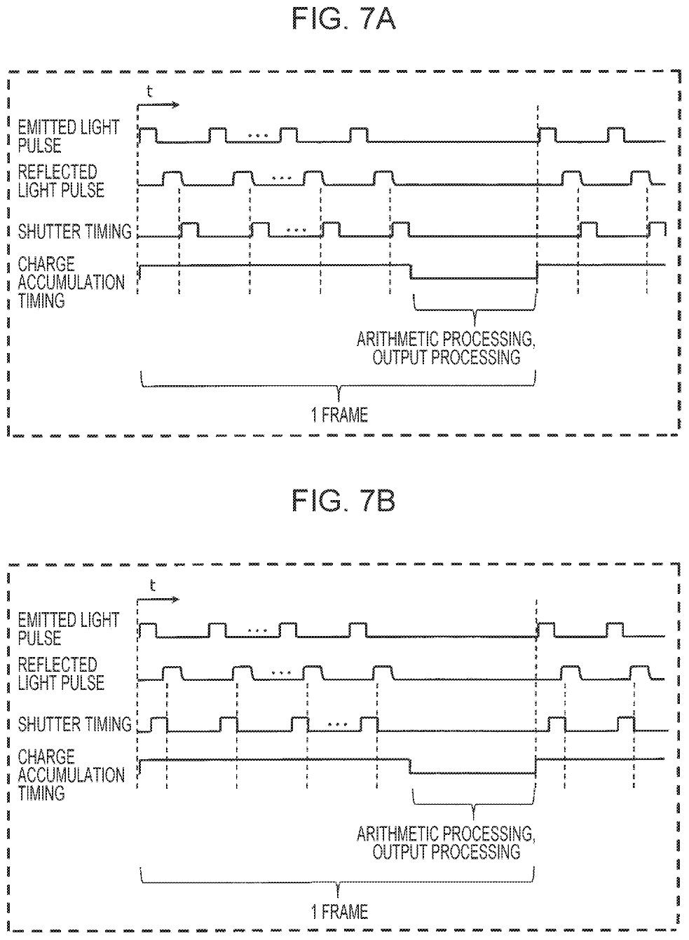

FIG. 7A is a timing diagram illustrating an example of an operation of detecting the internal scattered component I2. In this case, the light emitting device 110 repeatedly emits a light pulse during a one-frame period. The image sensor 120 opens the electronic shutter during a period where a rear end portion of each reflected light pulse reaches the image sensor 120. By this operation, the image sensor 120 accumulates a signal of the internal scattered component I2. After signal accumulation is performed a predetermined number of times, the image sensor 120 outputs a signal accumulated for each pixel as a detection signal. The output detection signal is processed by the processing circuit 134.

As described above, the control circuit 132 repeats the detection operation of causing the light emitting device 110 to emit a light pulse and causing the image sensor 120 to detect at least a part of a component after start of falling among components of the reflected light pulse and output a detection signal indicative of a spatial distribution of an intensity of an internal scattered component. By such an operation, the processing circuit 134 can generate and output distribution data indicative of a spatial distribution of a cerebral blood flow amount in the portion to be measured on the basis of the detection signal that is repeatedly output.

Next, an example of a method for detecting the surface reflected component I1 is described. The surface reflected component I1 includes information on a surface of the user 50. The information on the surface is, for example, information on a blood flow of a face and a scalp.

FIG. 7B is a timing diagram illustrating an example of an operation of detecting the surface reflected component I1. In a case where the surface reflected component I1 is detected, the image sensor 120 opens the shutter before each reflected light pulse reaches the image sensor 120 and closes the shutter before the rear end of the reflected light pulse reaches the image sensor 120. By thus controlling the shutter, it is possible to suppress inclusion of the internal scattered component I2 and increase a proportion of the surface reflected component I1. The timing at which the shutter is closed may be immediately after light reaches the image sensor 120. This makes it possible to perform signal detection in which a proportion of the surface reflected component I1 having a relatively short optical path length is increased. By acquiring a signal of the surface reflected component I1, it is possible to not only acquire a face image of the user 50, but also estimate a pulse or a degree of oxygenation of a blood flow in a surface layer of skin. As another method for acquiring the surface reflected component I1, the image sensor 120 may detect the whole reflected light pulse or may detect continuous light emitted from the light emitting device 110. An image sensor or a camera that acquires a face image of the user 50 may be provided separately from the image sensor 120. In this case, the image sensor 120 need not detect the surface reflected component I1.

The surface reflected component I1 may be detected by a device other than the measurement device 100 that acquires the internal scattered component I2. For example, another device such as a sphygmograph or a Doppler blood flow meter may be used. In this case, the other device is used in consideration of timing synchronization between devices, interference of light, and matching between detection portions. In a case where time-division imaging using the single measurement device 100 or the single sensor is performed as in the present embodiment, temporal and spatial deviations are less likely to occur. In a case where both of a signal of the surface reflected component I1 and a signal of the internal scattered component I2 are acquired by a single sensor, a component to be acquired may be switched every frame, as illustrated in FIGS. 7A and 7B. Alternatively, a component to be acquired may be switched at a high rate within one frame. In this case, a detection time difference between the surface reflected component I1 and the internal scattered component I2 can be reduced.

Furthermore, each of the signal of the surface reflected component I1 and the signal of the internal scattered component I2 may be acquired by using light of two wavelengths. For example, a light pulse having a wavelength of 750 nm and a light pulse having a wavelength of 850 nm may be used. This makes it possible to calculate a change in concentration of oxyhemoglobin and a change in concentration of deoxyhemoglobin from changes in amount of detected light of the wavelengths. In a case where the surface reflected component I1 and the internal scattered component I2 are acquired by using two wavelengths, a method of switching four kinds of charge accumulation at a high rate within one frame can be used, for example, as described with reference to FIGS. 3 to 5. By such a method, a temporal deviation of a detection signal can be reduced.

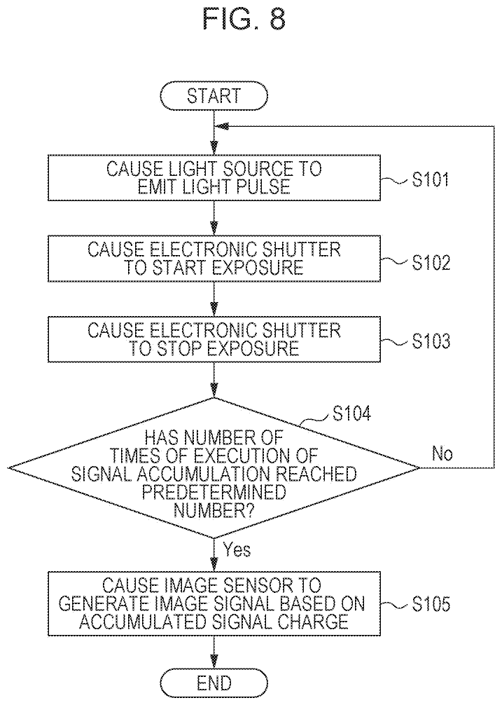

FIG. 8 is a flowchart illustrating an outline of an operation of controlling the light source 101 and the image sensor 120 by the control circuit 105. The following describes an example of an operation performed in a case where the internal scattered component I2 is detected by using light of a single wavelength. An operation of detecting the surface reflected component I1 is similar to the operation illustrated in FIG. 8 except for that timings of start and end of exposure relative to an emission timing are earlier. In a case where light of wavelengths is used, the operation illustrated in FIG. 8 is repeated for each wavelength.

In step S101, the control circuit 132 causes the light source 101 to emit a light pulse for a predetermined period. At this time, the electronic shutter of the image sensor 120 is not performing exposure. The control circuit 132 stops the electronic shutter from performing exposure until a period where a part of the light pulse is reflected by a surface of the forehead of the user 50 and reaches the image sensor 120 ends. In next step S102, the control circuit 132 causes the electronic shutter to start exposure at a timing at which a part of the light pulse scattered inside the forehead of the user 50 reaches the image sensor 120. After elapse of a predetermined period, in step S103, the control circuit 132 causes the electronic shutter to stop the exposure. In next step S104, the control circuit 105 determines whether or not the number of times of execution of the signal accumulation has reached a predetermined number. In a case where a result of this determination is No, steps S101 to S103 are repeated until the result of this determination becomes Yes. In a case where the result of the determination in step S104 is Yes, step S105 is performed, in which the control circuit 132 causes the image sensor 120 to generate and output a signal indicative of an image based on signal charges accumulated in the floating diffusion layers.

By the above operation, a light component scattered inside the measurement target can be detected with high sensitivity. Note that the emission and exposure need not necessarily be performed plural times and are performed plural times as needed.

### 2-2. Example of Signal Processing

Next, an example of signal processing performed by the processing circuit 134 is described.

The processing circuit 134 generates a cerebral blood flow signal of the user 50 on the basis of a detection signal (i.e., an internal image signal) of each pixel output from the image sensor 120. The cerebral blood flow signal includes, for example, at least one information among an oxyhemoglobin concentration, a deoxyhemoglobin concentration, and a total hemoglobin concentration, which is a sum of the oxyhemoglobin concentration and the deoxyhemoglobin concentration, in cerebral blood. The processing circuit 134 can obtain change amounts of concentrations of the oxyhemoglobin (HbO₂) and the deoxyhemoglobin (Hb) in blood from initial values by solving predetermined simultaneous equations on the basis of a signal value of the internal scattered component I2 measured for each pixel. The simultaneous equations are, for example, expressed by the following expressions (1) and (2):

$${ε}_{OXY}^{750} {Δ HbO}_{2} + {ε}_{deOXY}^{750} Δ Hb = - ln \frac{{I}_{now}^{750}}{{I}_{ini}^{750}} \qquad ( 1 )$$

$${ε}_{OXY}^{850} {Δ HbO}_{2} + {ε}_{deOXY}^{850} Δ Hb = - ln \frac{{I}_{now}^{850}}{{I}_{ini}^{850}} \qquad ( 2 )$$

where ΔHbO₂ and ΔHb represent change amounts of concentrations of HbO₂ and Hb in the blood from initial values, respectively, ε⁷⁵⁰_{OXY} and ε⁷⁵⁰_{deOXY} represent molar absorption coefficients of HbO₂ and Hb at the wavelength of 750 nm, respectively, ε⁸⁵⁰_{oxy} and ε⁸⁵⁰_{deOXY} represent molar absorption coefficients of HbO₂ and Hb at the wavelength of 850 nm, respectively, I⁷⁵⁰ᵢₙᵢ and I⁷⁵⁰_{now} represent detection intensities of the wavelength of 750 nm at an initial time and a detection time, respectively, and I⁸⁵⁰ᵢₙᵢ and I⁸⁵⁰_{now} represent detection intensities of the wavelength of 850 nm at an initial time and a detection time, respectively. The processing circuit 134 can calculate, for each pixel, the change amounts ΔHbO₂ and ΔHb of the concentrations of HbO₂ and Hb in the blood from the initial values, for example, on the basis of the expression (1) and (2). In this way, data of two-dimensional distributions of the change amounts of the concentrations of HbO₂ and Hb in the blood in the portion to be measured can be generated.

The processing circuit 134 can further calculate a degree of oxygen saturation of hemoglobin. The degree of oxygen saturation is a value indicative of a percentage of hemoglobin in the blood bound to oxygen. The degree of oxygen saturation is defined by the following expression:

degree of oxygen saturation=C(HbO₂)/[C(HbO₂)+C(Hb)]×100(%)

where C (Hb) is a concentration of the deoxyhemoglobin and C (HbO₂) is a concentration of the oxyhemoglobin. The living body includes components that absorb red light and near-infrared light in addition to blood. However, a temporal fluctuation in light absorption rate is mainly caused by hemoglobin in arterial blood. Therefore, a degree of oxygen saturation in blood can be measured with high accuracy on the basis of a fluctuation in absorption rate.

Light that has reached the brain also passes through the scalp and face surface. Accordingly, a fluctuation in blood flow in the scalp and face is also detected. To remove or reduce influence of the fluctuation in blood flow in the scalp and face, the processing circuit 134 may perform processing of subtracting the surface reflected component I1 from the internal scattered component I2 detected by the image sensor 120. This makes it possible to acquire pure cerebral blood flow information excluding blood flow information of the scalp and face. The subtracting method can be, for example, a method of multiplying a signal of the surface reflected component I1 by a coefficient decided in consideration of an optical path length difference and subtracting a value thus obtained from the signal of the internal scattered component I2. This coefficient can be, for example, calculated by simulation or an experiment on the basis of an average of optical constants of general human heads. Such subtracting processing can be easily performed in a case where measurement is performed by using light of a single wavelength by a single measurement device. This is because it is easier to reduce temporal and spatial deviations and achieve matching between characteristics of a scalp blood flow component included in the internal scattered component I2 and characteristics of the surface reflected component I1.

The skull is present between the brain and the scalp. Accordingly, a two-dimensional distribution of a cerebral blood flow and a two-dimensional distribution of a scalp and face blood flow are independent. Therefore, the two-dimensional distribution of the internal scattered component I2 and the two-dimensional distribution of the surface reflected component I1 may be separated on the basis of a signal detected by the image sensor 120 by using a statistical method such as independent component analysis or principal component analysis.

The processing circuit 134 may generate, as brain activity data, moving image data indicative of spatial and temporal fluctuations of a cerebral blood flow signal indicative of the oxyhemoglobin concentration, the deoxyhemoglobin concentration, the total hemoglobin concentration, the blood oxygen saturation level, or the like. Alternatively, the processing circuit 134 may generate, as brain activity data, data indicative of a psychological state or a physical state of the user 50 estimated from a cerebral blood flow signal.

It is known that there is a close relationship between a change in cerebral blood flow amount or component in blood such as hemoglobin and neural activity of a person. For example, when activity of nerve cells changes in accordance with a change in feeling of a person, a cerebral blood flow amount or a component in blood changes. Accordingly, in a case where biological information such as a change in cerebral blood flow amount or component in blood can be measured, a user's psychological state or physical state can be estimated. The user's psychological state can include, for example, a state such as a mood, a feeling, a health condition, or a sense of temperature. The mood can include, for example, a mood such as a good mood or a bad mood. The feeling can include, for example, a feeling such as a sense of safety, a sense of anxiety, sadness, or anger. The health condition can include, for example, a condition such as a good condition or a fatigued condition. The sense of temperature can include, for example, a sense such as hot, cold, or hot and humid. The psychological state can also include derivatives of these, specifically, indices indicative of a degree of brain activity such as a degree of interest, a degree of proficiency, a level of learning, and a degree of concentration. Furthermore, a physical state such as a degree of fatigue, sleepiness, or a degree of alcohol intoxication may be estimated. In the present specification, such data related to a cerebral blood flow is collectively referred to as “brain activity data”.

A method for estimating a brain activity amount such as a degree of concentration on the basis of a cerebral blood flow signal is, for example, disclosed in Patent Literature 2. The entire disclosure of the Patent Literature 2 is incorporated herein.

The processing circuit 134 according to the present embodiment decides which of the first region and the second region of the forehead of the user 50 is used on the basis of a face image indicated by a pulse front end signal of each pixel of the image sensor 120 and output brain activity data based on a signal in the decided region. The processing circuit 134 detects a change in shape of skin of a forehead portion of the user 50 from the face image, selects the first region or the second region as a target region on the basis of an amount of the change, and generates brain activity data on the basis of a detection signal (i.e., a pulse rear end signal) in the selected target region. Note that the target region may be decided by using an image signal indicative of the face of the user 50 acquired by a sensor different from the image sensor 120 instead of the face image indicated by the pulse front end signal of each pixel output from the image sensor 120.

The measurement device 100 may repeat emission of a light pulse, detection of a reflected light pulse, generation of a cerebral blood flow signal, and generation and output of brain activity data on a predetermined cycle. This makes it possible to generate a moving image indicative of the face and a state of a cerebral blood flow of the user 50. The measurement device 100 basically outputs brain activity data based on a detection signal in the first region of high sensitivity, but outputs brain activity data based on a detection signal in the second region that is less influenced by noise in a case where a preset condition is satisfied. For example, in a case where an amount of movement of skin of the forehead portion of the user 50 detected from the face image signal is larger than a predetermined amount, brain activity data based on a detection signal in the second region is output. At a timing at which a state switches from a first state in which brain activity data based on a detection signal in the first region is output to a second state in which brain activity data based on a detection signal in the second region is output, the processing circuit 134 may output a signal indicative of the switching of the state. The signal may be, for example, sent to the display 300, and an image indicative of the switching of the state may be displayed on the display 300. The signal indicative of the switching between the first state and the second state may be, for example, used for weighting in signal processing for estimating an internal state such as a state of brain activity of the user 50. In a case where the above condition ceases to be satisfied in the second state in which brain activity data based on a detection signal in the second region is output, the processing circuit 134 switches the second state to the first state in which brain activity data based on a detection signal in the first region is output. Also in this case, the processing circuit 134 may output a signal indicative of the switching from the second state to the first state. To ensure continuity of brain activity data at the time of switching between the first state and the second state, a baseline of data after the switching may be reset by using a value immediately before the switching.

### 2-3. Example of Region Switching Processing

Next, an example of a method for generating brain activity data of the user 50 by selecting an appropriate region from among regions is described. The processing circuit 134 according to the present embodiment measures an amount of movement of an eyebrow of the user 50 on the basis of a surface image signal. The amount of movement of the eyebrow is, for example, a distance of movement of a feature point of the eyebrow, that is, an amount of displacement of the feature point from a reference position. The processing circuit 134 decides a region from among regions including the first region and the second region on the basis of the amount of movement of the eyebrow of the user 50 and outputs brain activity data based on a measurement signal in the decided region.

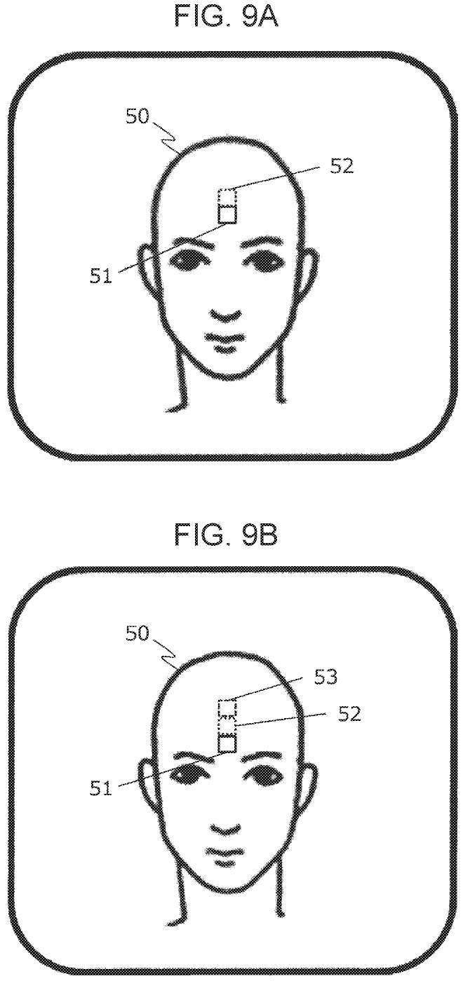

FIGS. 9A to 9D illustrate an example of the regions that can be used for generation of brain activity data to be output. In the example illustrated in FIG. 9A, a first region 51 is set at a predetermined position of the forehead of the user 50, and a second region 52 is set on an upper side relative to the first region 51. A position of high measurement sensitivity is selected as the position of the first region 51. A position that is lower in sensitivity than the first region 51 but is less influenced by noise resulting from a change in shape of skin is selected as the position of the second region 52. In this example, the second region 52 is set directly above the first region 51 so that the second region 52 is less influenced by a change in shape of skin. The second region 52 need not be adjacent to the first region 51 directly above the first region 51 and may be apart from the first region 51. Furthermore, the first region 51 and the second region 52 may be different in position in a left-right direction.

As illustrated in FIG. 9B, three or more regions may be set. In the example of FIG. 9B, the first region 51, the second region 52, and a third region 53 arranged in an up-down direction are set. The second region 52 is set directly above the first region 51, and the third region 53 is set directly above the second region 52. In this example, the processing circuit 134 can be, for example, configured to output brain activity data based on a detection signal in a region located higher as an amount of movement of the eyebrow becomes larger. For example, the processing circuit 134 may output brain activity data based on a detection signal in the first region 51 in a case where the amount of movement of the eyebrow is smaller than a first threshold value, may output brain activity data based on a detection signal in the second region 52 in a case where the amount of movement of the eyebrow is equal to or larger than the first threshold value and smaller than a second threshold value, and may output brain activity data based on a detection signal in the third region 53 in a case where the amount of movement of the eyebrow is equal to or larger than the second threshold value. By such processing, it is possible to select a more appropriate region in accordance with a degree of change in shape of skin and output brain activity data. Note that the first region 51, the second region 52, and the third region 53 need not be adjacent to each other, and may be apart from each other. Furthermore, the first region 51, the second region 52, and the third region 53 may be different in position in the left-right direction.

As illustrated in FIG. 9C, a part of the first region 51 and a part of the second region 52 may overlap each other. By thus setting the regions, the second region 52 can be set even in a case where there is no sufficient region above the first region 51. Also in a case where three or more regions are set, these regions may overlap each other.

As illustrated in FIG. 9D, a size of the first region 51 and a size of the second region 52 may be different. In the example illustrated in FIG. 9D, an area of the second region 52 is larger than an area of the first region 51. Although the second region 52 located on an upper side is relatively low in detection sensitivity, a sufficient signal amount can be observed by thus setting the measurement area large. In this case, for example, the processing circuit 134 generates brain activity data by using an arithmetic mean value of signals of pixels in each region.

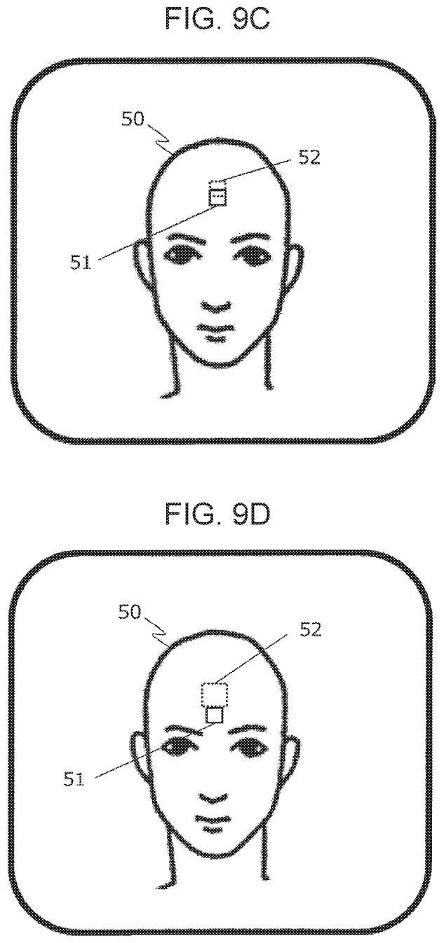

FIG. 10 is a flowchart illustrating an example of processing for generating brain activity data of the user 50. The measurement device 100 generates brain activity data by performing operations in steps S101 to S110 illustrated in FIG. 10.

In step S101, the measurement device 100 perform initial setting necessary for measurement. The initial setting includes a step in which the control circuit 132 adjusts an emission timing of a light pulse from the light emitting device 110 and a shutter timing of the image sensor 120 to optimum timings in accordance with a distance between the measurement device 100 and the user 50. The initial setting includes a step in which the control circuit 132 causes the image sensor 120 to output a surface image signal including the surface reflected component I1 and a step in which the processing circuit 134 decides a position of an eyebrow of the user 50 in an initial state and records the position in the memory 136. Measurement (steps S102 to S110) of a cerebral blood flow can be performed after completion of the initial setting.

In step S102, the control circuit 132 causes the image sensor 120 to output an internal image signal indicative of an intensity distribution of the internal scattered component I2.

In step S103, the control circuit 132 causes the image sensor 120 to output a surface image signal indicative of an intensity distribution of the surface reflected component I1. Note that although a surface image signal is used in the present embodiment, an image signal acquired by a sensor or a camera provided separately from the image sensor 120 may be used instead of the surface image signal. Step S102 and step S103 may be performed in a reverse order or step S102 and step S103 may be performed concurrently.

In step S104, the processing circuit 134 decides regions of the forehead from a surface image output from the image sensor 120. The regions of the forehead can be, for example, decided by using a known face recognition technique. The processing circuit 134 extracts pixel regions to be measured from among the regions of the forehead on the basis of the position of the eyebrow decided in step S101. For example, two or more regions including the first region 51 and the second region 52 are set, as in the examples illustrated in FIGS. 9A to 9D. The regions need not be adjacent to each other, and each of the regions can have any shape. Even in a case where these regions have different shapes and areas, the processing circuit 134 can properly generate brain activity data corresponding to each region by using an arithmetic mean value of signal values of pixels included in each region.

In step S105, the processing circuit 134 specifies a position of the eyebrow from the surface image signal and calculates an absolute value of a difference from the position of the eyebrow in the initial state recorded in the memory 136. This absolute value of the difference is referred to as an amount of displacement of the eyebrow. The processing for specifying the position of the eyebrow from the surface image signal can be, for example, performed by using a known image processing method such as feature point extraction using edge detection. The region of the eyebrow includes a larger number of components of a high spatial frequency than the region of the forehead in the image. It is therefore possible to easily extract feature points by using the region of the eyebrow.

In step S106, the processing circuit 134 determines whether or not the amount of displacement of the eyebrow is equal to or smaller than a threshold value. The threshold value can be, for example, set to any value included in a range of 1 mm to 10 mm. The threshold value is set to an appropriate value in accordance with a degree of requested accuracy of brain activity data.

In a case where the amount of displacement of the eyebrow is equal to or smaller than the threshold value, step S107 is performed. In step S107, the processing circuit 134 generates brain activity data by using a part of the internal image signal that corresponds to the first region 51.

In a case where the amount of displacement of the eyebrow is larger than the threshold value, step S108 is performed. In step S108, the processing circuit 134 generates brain activity data by using a part of the internal image signal that corresponds to the second region 52.

In step S109, the processing circuit 134 outputs brain activity data. The brain activity data can be, for example, sent to the display 300, and information indicative of a state of brain activity of the user 50 can be displayed. Alternatively, the brain activity data may be sent to another device (not illustrated). For example, the brain activity data may be sent to a computer that controls an apparatus in accordance with the state of brain activity indicated by the brain activity data.

Sensitivity of detection of a change amount of a cerebral blood flow is higher in the first region 51 than in the second region 52. On the other hand, in the first region 51, a shape of a surface layer of skin greatly changes due to influence of a change in facial expression, and resulting noise is large. Therefore, the processing circuit 134 according to the present embodiment basically outputs brain activity data based on a detection signal in the first region 51, and outputs brain activity data based on a detection signal in the second region 52 in a case where a shape of a surface layer of skin of the forehead changes, for example, in accordance with a change in facial expression. By such an operation, not only measurement sensitivity can be maintained, but also measurement less influenced by noise can be performed. The processing circuit 134 may determine whether or not the first region 51 is suitable as a target region for measurement of a cerebral blood flow, and output brain activity data based on a detection signal in the first region 51 in a case where the first region 51 is suitable. Whether or not the first region 51 is suitable as a target region for measurement of a cerebral blood flow can be determined on the basis of whether or not an amount of displacement of the eyebrow from the reference position is equal to or larger than a threshold value. It may be determined that the first region 51 is suitable as the target region in a case where the amount of displacement of the eyebrow from the reference position is smaller than the threshold value. In a case where the amount of displacement of the eyebrow from the reference position is equal to or larger than the threshold value, it may be determined that the first region 51 is not suitable as the target region, and brain activity data based on a detection signal in the second region 52 may be output. Furthermore, during a period in which brain activity data based on a detection signal in the second region 52 is being output, it may be determined again whether or not the first region 51 is suitable as the target region. In a case where it is determined that the first region 51 is suitable as the target region, brain activity data may be output after switching the target region from the second region 52 to the first region 51. Furthermore, it may be determined whether or not the first region 51 is suitable as the target region for measurement of a cerebral blood flow on the basis of a change in oxyhemoglobin concentration and a change in deoxyhemoglobin concentration, as described later.

In step S110, it is determined whether or not measurement for a predetermined period has been completed. This determination may be performed by the measurement device 100 itself or may be performed by a computer connected to the measurement device 100.

The “predetermined period” can be, for example, a period necessary for estimating a psychological state such as a degree of concentration of the user 50. Alternatively, in a case where measurement is performed while the user 50 is performing a series of tasks or operations, the “predetermined period” can be a period to the end of the series of tasks or operations. The series of tasks or operations can be, for example, study, a puzzle, office work, driving of an automobile, or an operation of a gaming console.

In a case where the measurement for the predetermined period has not been completed yet, the measurement device 100 repeats the sequence from step S102 to step S110. In a case where the measurement for the predetermined period has been completed, the measurement device 100 finishes the measurement.

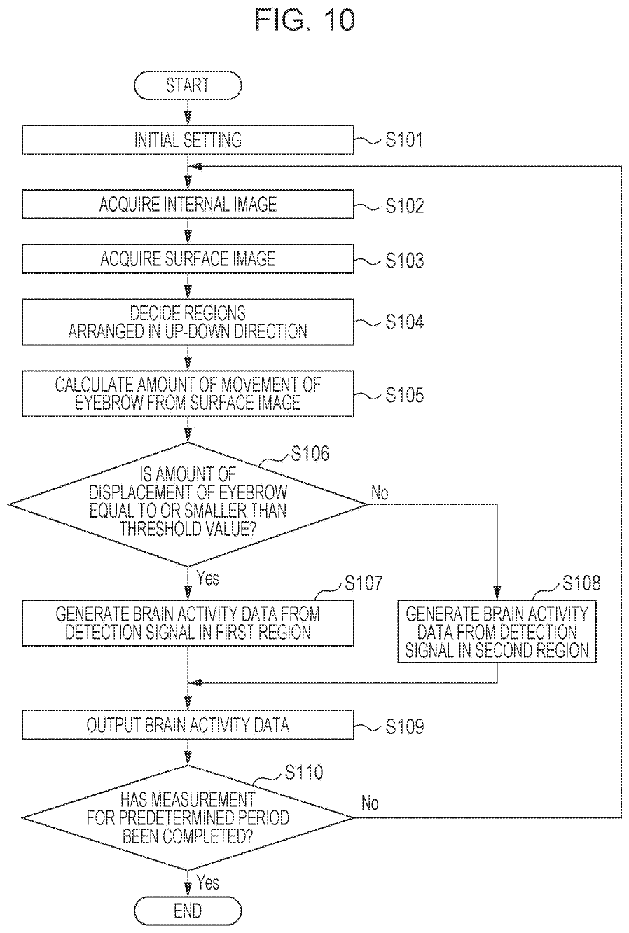

FIG. 11 illustrates an example of an operation of switching the target region in accordance with a change in facial expression of the user 50. In each of the graphs illustrated in FIG. 11, the horizontal axis represents a frame or transition of time. (a) of FIG. 11 schematically illustrates an example of an image indicative of an intensity distribution of the surface reflected component I1. (b) FIG. 11 illustrates an example of a temporal change of an amount of displacement of the eyebrow. (c) of FIG. 11 illustrates an example of a temporal change of a selected target region. (d) of FIG. 11 illustrates an example of transition of a value of output data in each frame. In this example, a cerebral blood flow signal in the first region or the second region of the forehead is output as brain activity data. The cerebral blood flow signal in this example is a signal indicative of a change amount of a concentration of oxyhemoglobin (HbO₂) in cerebral blood from a reference value, but is not limited to this.

FIG. 11 illustrates five frames, which are given numbers 1 to 5. These frame numbers are merely illustrative, and actually, one or more frames may be interposed between the illustrated frames given numbers. The number of times of output of data per unit time is referred to as a frame rate. The frame rate can be, for example, set to a value within a range of 1 frame per second (fps) to 30 fps. A rate of measurement of the amount of displacement of the eyebrow and a rate of generation of brain activity data may be different. The cerebral blood flow amount gradually changes over 1 second to several seconds. On the other hand, the amount of displacement of the eyebrow changes faster than the cerebral blood flow amount. Therefore, the rate of measurement of the amount of displacement of the eyebrow may be set higher than a rate of measurement of a cerebral blood flow amount to detect the amount of displacement of the eyebrow more precisely.

In the example illustrated in FIG. 11, in the third and fourth frames, the amount of displacement of the eyebrow measured by the processing circuit 134 is larger than the threshold value due to a change in facial expression of the user 50. There is a correlation between the amount of displacement of the eyebrow and irregularity of generated brain activity data. Therefore, during the period where the amount of displacement of the eyebrow is larger than the threshold value, the processing circuit 134 stops output of brain activity data based on a detection signal in the first region 51, and outputs brain activity data based on a detection signal in the second region 52. This period is referred to as a switching period. When the amount of displacement of the eyebrow becomes lower than the threshold value, the processing circuit 134 stops output of brain activity data based on a detection signal in the second region 52, and outputs brain activity data based on a detection signal in the first region 51 again. Therefore, in the example illustrated in FIG. 11, brain activity data based on a detection signal in the first region 51 is output in the frames 1, 2, and 5, and brain activity data based on a detection signal in the second region 52 is output in the frames 3 and 4.

As described above, according to the present embodiment, brain activity data is generated on the basis of a detection signal in the second region 52 located on an upper side during a period where noise included in a detection signal in the first region 51 is large, for example, due to a change in facial expression of the user 50. By performing such an operation, brain activity data can be measured with high accuracy in a non-contact manner without prompting the user 50 to perform measurement again. It is therefore possible to provide the measurement device 100 that allows the user 50 to routinely measure his or her brain activity state.

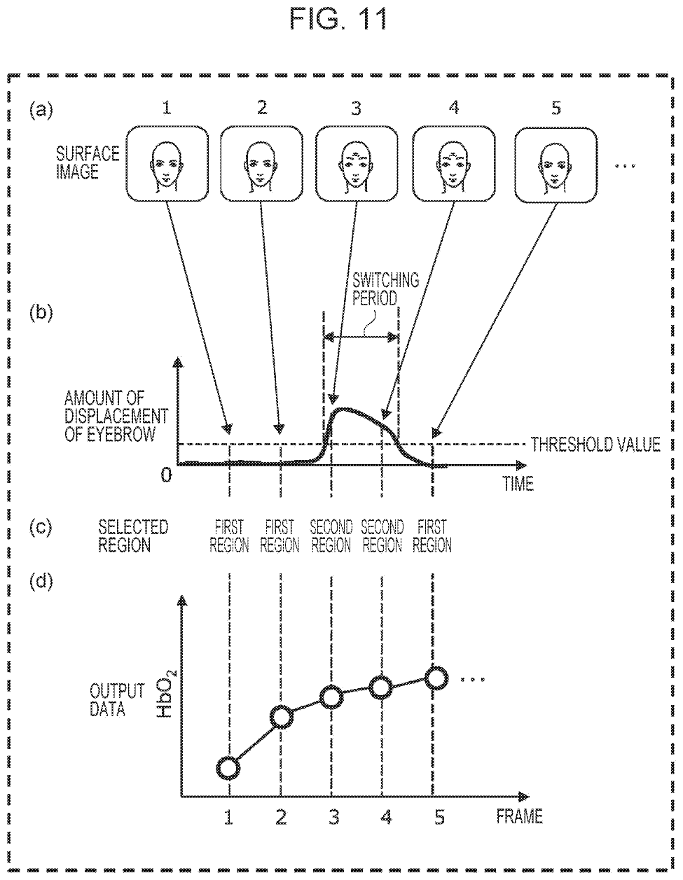

Although the period where the amount of displacement of the eyebrow is larger than the threshold value matches the switching period where brain activity data based on a detection signal in the second region is output in the example illustrated in FIG. 11, these periods need not strictly match each other. For example, relatively short periods before and after the period where the amount of displacement of the eyebrow is larger than the threshold value may be included in the switching period, as illustrated in FIGS. 12A and 12B.

FIG. 12A illustrates an example of a relationship between the amount of displacement of the eyebrow and the switching period. In this example, not only the period where the amount of displacement of the eyebrow is larger than the threshold value, but also a period df1 before the amount of displacement of the eyebrow becomes larger than the threshold value and a period dr1 after the amount of displacement of the eyebrow becomes smaller than the threshold value are included in the switching period. In this case, brain activity data is generated later than a cerebral blood flow signal by a period longer than the periods df1 and dr1. In this example, the periods df1 and dr have an identical length, and each period can be, for example, a period corresponding to 0.5 frames. The switching period may be a period including any one of the periods df1 and dr. As described above, the processing circuit 134 may set, as the switching period, a period including not only the period where the amount of displacement of the eyebrow is larger than the threshold value, but also at least one of the period df1 before start of this period and the period dr1 after end of this period.

FIG. 12B illustrates another example of a relationship between the amount of displacement of the eyebrow and the switching period. In this example, a period dr2 after the end of the period where the amount of displacement of the eyebrow is larger than the threshold value is longer than a period dr2 before the start of this period. The period df2 can be, for example, a period corresponding to 0.5 frames, and the period dr2 can be, for example, a period corresponding to 4.5 frames. In a case where the processing circuit 134 performs processing of smoothing a temporal fluctuation of a signal by applying a low-pass filter based on a moving average to a cerebral blood flow signal, influence of sudden noise remains in subsequent several frames. In such a case, the remaining influence of the noise can be effectively suppressed by including, in the switching period, the relatively long period after the end of the period where the amount of displacement of the eyebrow is larger than the threshold value, as illustrated in FIG. 12B.

### 3. Modification

Although a target region is decided from among regions on the basis of an amount of displacement of an eyebrow from an initial position in the present embodiment, the target region may be decided by another method. For example, the target region may be decided on the basis of a rate of change of the eyebrow. The rate of change of the eyebrow is an amount of change of the eyebrow between frames. A rate of change of the eyebrow in one frame can be, for example, an amount of displacement from a position of the eyebrow in an immediately preceding frame. The processing circuit 134 may determine that a change in shape of skin has occurred in a frame in which an absolute value of the rate of change of the eyebrow is larger than a threshold value. For example, the processing circuit 134 may output brain activity data based on a detection signal in the first region 51 in a frame in which the absolute value of the rate of change of the eyebrow is not larger than the threshold value and may output brain activity data based on a detection signal in the second region 52 located on an upper side in a frame in which the absolute value of the rate of change of the eyebrow is larger than the threshold value.

FIG. 12C illustrates an example of a relationship between a rate of change of the eyebrow and the switching period. In this example, the switching period starts at a timing at which the rate of change of the eyebrow becomes higher than a positive threshold value, and after the rate of change of the eyebrow becomes lower than the positive threshold value and becomes lower than a negative threshold value, the switching period ends at a timing at which the rate of change of the eyebrow becomes higher than the negative threshold value. Conversely, the switching period may start at a timing at which the rate of change of the eyebrow becomes lower than the negative threshold value, and after the rate of change of the eyebrow becomes higher than the negative threshold value and becomes higher than the positive threshold value, the switching period may end at a timing at which the rate of change of the eyebrow becomes lower than the positive threshold value.

The processing circuit 134 may calculate an amount of displacement of the eyebrow or a rate of change of the eyebrow on the basis of an internal image signal indicative of an intensity distribution of the internal scattered component I2 instead of a surface image signal indicative of an intensity distribution of the surface reflected component I1. Since the internal image signal also includes information on an outer shape of the face of the user 50, an amount of displacement of the eyebrow can be calculated on the basis of a temporal change of the internal image signal. However, since the surface image signal typically has higher luminance than the internal image signal, use of the surface image signal is often advantageous in terms of an SN ratio.

The target region may be decided on the basis of tendency of a change of a cerebral blood flow signal instead of calculating an amount of displacement or a rate of change of the eyebrow of the user 50 from an image signal and deciding the target region on the basis of the amount or the rate. For example, the processing circuit 134 may decide the target region on the basis of a rate of change of a cerebral blood flow signal. The cerebral blood flow signal is, for example, a signal indicative of an amount of change of an oxyhemoglobin concentration, a deoxyhemoglobin concentration, or a total hemoglobin concentration, which is a sum of the oxyhemoglobin concentration and the deoxyhemoglobin concentration, from a reference value. A cerebral blood flow changes gradually over 1 second to several seconds. On the other hand, a change in shape and a change in luminance of skin caused by a change in movement or facial expression of the user 50 influence a measurement value of the cerebral blood flow at a rate higher than the cerebral blood flow. Therefore, the processing circuit 134 can determine effectiveness of a detection signal in the first region 51 on the basis of a rate of change of the cerebral blood flow signal. The processing circuit 134 may stop output of brain activity data based on a detection signal in the first region 51 and output brain activity data based on a detection signal in the second region 52 in a case where an absolute value of the rate of change of the cerebral blood flow signal is larger than a threshold value. By performing such an operation, the measurement device 100 can output more accurate brain activity data while maintaining sensitivity of measurement.

The processing circuit 134 may determine effectiveness of a detection signal in the first region 51 on the basis of a phase of a change in oxyhemoglobin concentration and a phase of a change in deoxyhemoglobin concentration. The processing circuit 134 can generate a cerebral blood flow signal indicative of a temporal change in oxyhemoglobin concentration and a temporal change in deoxyhemoglobin concentration. In a case where the oxyhemoglobin concentration increases due to a change in cerebral blood flow, the deoxyhemoglobin concentration decreases. Conversely, in a case where the oxyhemoglobin concentration decreases, the deoxyhemoglobin concentration increases. That is, a phase of a change in oxyhemoglobin concentration resulting from a cerebral blood flow and a phase of a change in deoxyhemoglobin concentration resulting from a cerebral blood flow are reverse to each other. On the other hand, a phase of a change in oxyhemoglobin concentration resulting from a change in shape of skin and a phase of a change in deoxyhemoglobin concentration resulting from a change in shape of skin are identical to each other. Therefore, the processing circuit 134 may output brain activity data based on a detection signal of the first region 51 in a case where a phase of a change in oxyhemoglobin concentration and a phase of a change in deoxyhemoglobin concentration in the first region 51 are reverse to each other and output brain activity data based on a detection signal in the second region 52 by stopping output of brain activity data in the first region in a case where the phases are identical. In other words, the processing circuit 134 may select the first region 51 as the target region during a period where one of the oxyhemoglobin concentration and the deoxyhemoglobin concentration in the first region 51 increases and the other one of the oxyhemoglobin concentration and the deoxyhemoglobin concentration in the first region 51 decreases, and may select the second region 52 as the target region during a period where both of the oxyhemoglobin concentration and the deoxyhemoglobin concentration in the first region 51 increase or decrease. Even by such an operation, it is possible to output more accurate brain activity data while maintaining sensitivity of measurement.

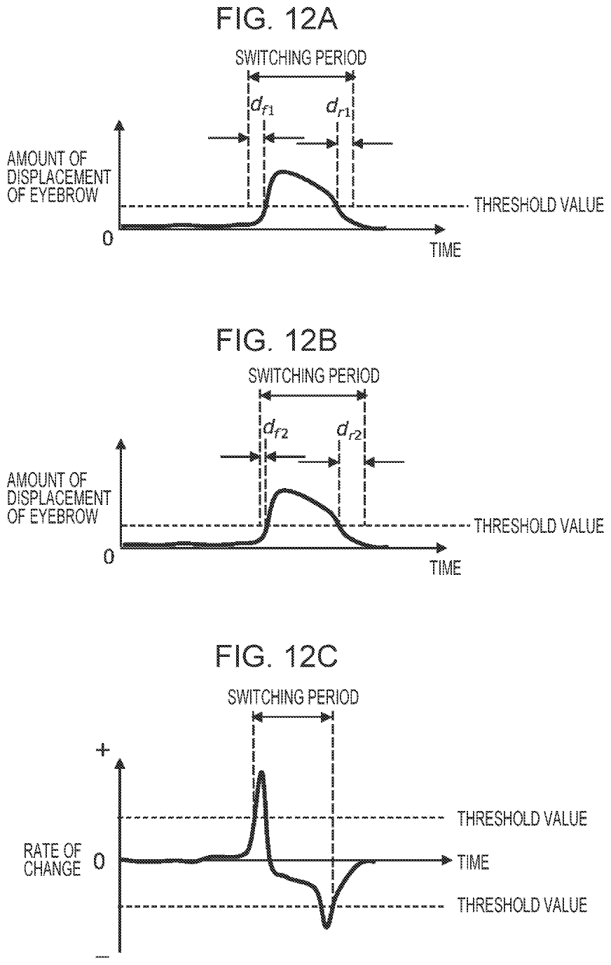

FIG. 13 is a flowchart illustrating an example of processing for deciding the target region on the basis of a phase of a temporal change in oxyhemoglobin concentration and a phase of a temporal change in deoxyhemoglobin concentration. In the flowchart illustrated in FIG. 13, operations in steps S101 to S104 and S107 to S110 are similar to those in corresponding steps in the flowchart illustrated in FIG. 10. However, the operation of deciding a position of an eyebrow in an initial state from a surface image in the initial setting in step S101 can be omitted. The following describes differences from the operation illustrated in FIG. 10.

In the example of FIG. 13, steps S115, S116, and S117 are performed after step S104 instead of steps S105 and S106.

In step S115, the processing circuit 134 calculates change amounts of the oxyhemoglobin concentration and the deoxyhemoglobin concentration from reference values on the basis of a detection signal in the first region 51 included in an internal image. This calculation is performed on the basis of the expressions (1) and (2) described above. The reference values can be, for example, initial values of the concentrations at the start of measurement. The processing circuit 134 further calculates absolute values of change rates of the oxyhemoglobin concentration and the deoxyhemoglobin concentration and phases of the changes of the oxyhemoglobin concentration and the deoxyhemoglobin concentration. The change rates can be, for example, calculated by dividing amounts of changes of the concentrations occurring during successive frames by a period of the frames.

In step S116, the processing circuit 134 determines whether or not an absolute value of a change rate of any one of the hemoglobin concentrations is equal to or smaller than a threshold value. The threshold value can be, for example, a value within a range from 0.2 mM·mm/s to 1.0 mM·mm/s. In a case where an absolute value of a change rate of at least one of the oxyhemoglobin concentration and the deoxyhemoglobin concentration is equal to or smaller than the threshold value, step S117 is performed. In a case where absolute values of change rates of both of the oxyhemoglobin concentration and the deoxyhemoglobin concentration are larger than the threshold value, step S108 is performed.

In step S117, the processing circuit 134 determines whether or not a phase of the change of the oxyhemoglobin concentration and a phase of the change of the deoxyhemoglobin concentration are reverse to each other. In a case where the phases are reverse to each other, step S107 is performed. In a case where the phases are identical, step S108 is performed.

The subsequent operations are similar to those illustrated in FIG. 10. By the operation illustrated in FIG. 13, it is possible to output more accurate brain activity data while maintaining sensitivity of measurement.

### Second Embodiment

Next, a biological measurement device according to a second embodiment is described.

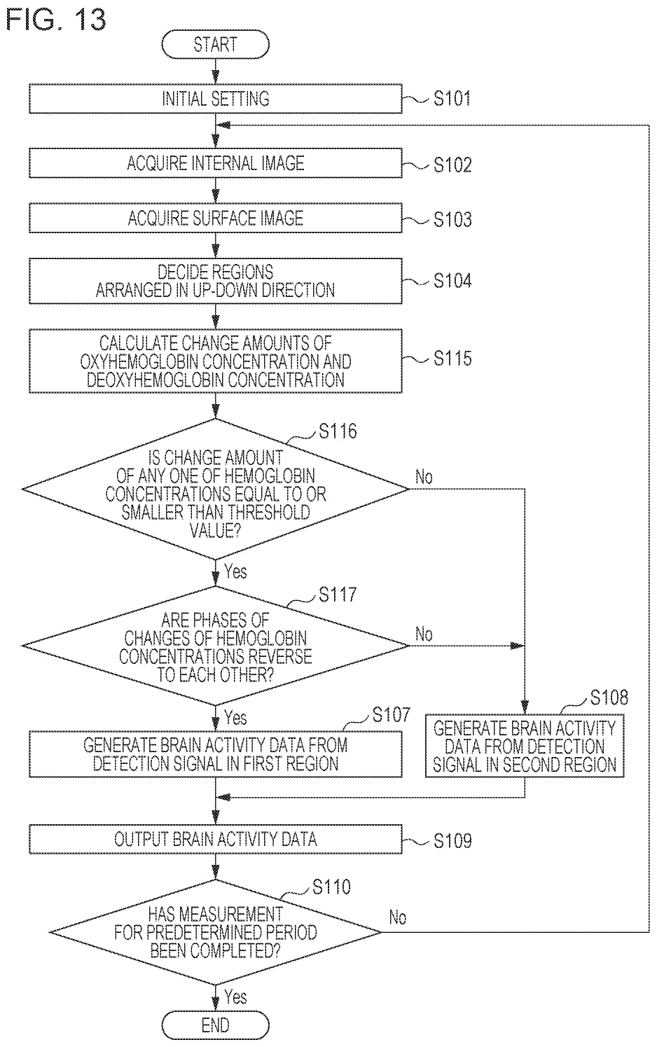

FIG. 14 schematically illustrates a configuration of a biological measurement device 200 according to the present embodiment. The biological measurement device 200 is a contact type NIRS device, and includes an NIRS sensor 250, a processing device 230, and a camera 270. The processing device 230 is connected to the NIRS sensor 250 and the camera 270. The processing device 230 includes a control circuit 232, a processing circuit 234, and a memory 236. The control circuit 232 controls the NIRS sensor 250. The processing circuit 234 generates and outputs brain activity data on the basis of signals output from the NIRS sensor 250 and the camera 270. The NIRS sensor 250 has a band-shaped structure and is wound around the forehead of a user 50.

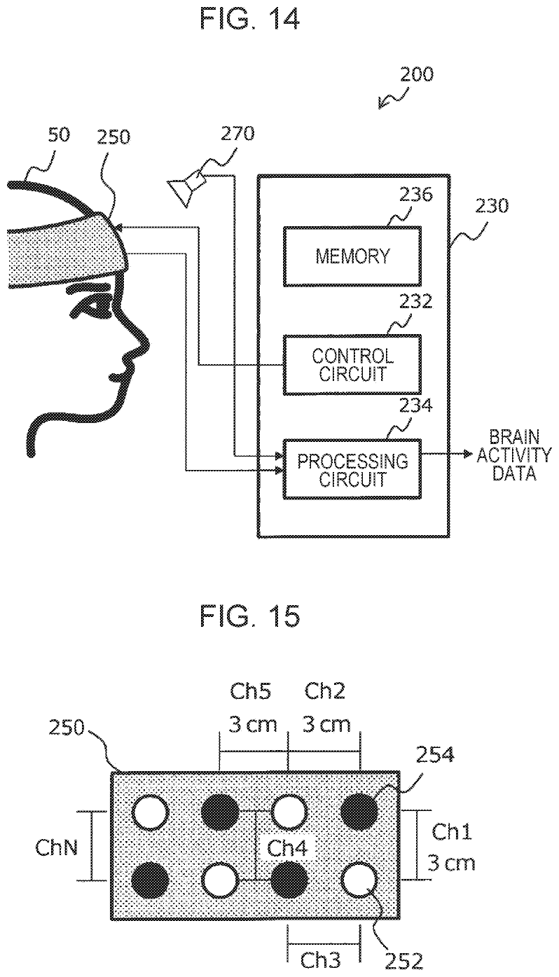

FIG. 15 schematically illustrates an example of a configuration of the NIRS sensor 250 on a rear side, that is, a side close to the forehead. The NIRS sensor 250 includes light sources 252 and photodetectors 254. In the example illustrated in FIG. 15, the light sources 252 and the photodetectors 254 are arranged in a matrix. Although four light sources 252 and four photodetectors 254 are provided in this example, the number of light sources 252 and the number of photodetectors 254 can be any numbers.

In the example illustrated in FIG. 15, each of the photodetectors 254 is disposed away by 3 cm from a position of an adjacent light source 252 in a lateral or longitudinal direction. A pair of the light source 252 and the photodetector 254 that are adjacent in the lateral or longitudinal direction is referred to as a “channel (Ch)”. In FIG. 15, channels (Ch1, Ch2, . . . , and CnN) are illustrated. A center-to-center distance between the light source 252 and the photodetector 254 in each channel is 3 cm in the example illustrated in FIG. 15, but is not limited to this.

The light sources 252 and the photodetectors 254 operate in response to a command from the control circuit 232. Each of the light sources 252 emits, for example, a near-infrared ray included in a wavelength range equal to or longer than 650 nm and equal to or shorter than 950 nm. Each of the photodetectors 254 detects scattering light that is light emitted from a corresponding one of the light sources 252 and scattered by an internal tissue of a forehead portion of the user 50 and outputs a detection signal according to an intensity of the scattering light.

The NIRS sensor 250 may emit light of two or more wavelengths included in the wavelength range. For example, the light sources 252 may include a light source that emits light having a wavelength equal to or longer than 650 nm and shorter than 805 nm and a light source that emits light having a wavelength longer than 805 nm and equal to or shorter than 950 nm. By irradiating the forehead of the user 50 with light in such wavelength ranges and detecting light that has passed through a body tissue, a change in oxygenation state of hemoglobin in blood of the brain can be detected on the basis of an amount of decrease of the light. Since the NIRS sensor 250 is mounted on the forehead, a change in blood flow amount in the frontal lobe can be detected.

In the example illustrated in FIG. 15, for example, a region of the forehead measured by the light source 252 and the photodetector 254 of Ch3 can be set as a “first region”, and a region of the forehead measured by the light source 252 and the photodetector 254 of Ch2 located above Ch3 can be set as a “second region”. Alternatively, the region of the forehead measured by the light source 252 and the photodetector 254 of Ch3 can be set as a “first region”, and a region of the forehead measured by the light source 252 and the photodetector 254 of Ch1 or Ch4 located obliquely above Ch3 can be set as a “second region”. As described above, the first region and the second region according to the present embodiment are decided as a combination of channels, and various methods can be used to select the first region and the second region.

The camera 270 includes an image sensor and outputs an image signal indicative of an image including the face of the user 50. The image signal is generated at a predetermined frame rate and is sequentially sent to the processing circuit 234.

The processing circuit 234 detects an amount of change in facial expression of the user 50 from the image signal acquired from the camera 270. For example, the processing circuit 234 detects an amount or rate of displacement of one or more feature points of the eyebrow of the user 50 by a method similar to the first embodiment, decides which of the first region and the second region located on an upper side relative to the first region is used in accordance with the amount or rate, and generates and outputs brain activity data on the basis of a detection signal in the decided region.

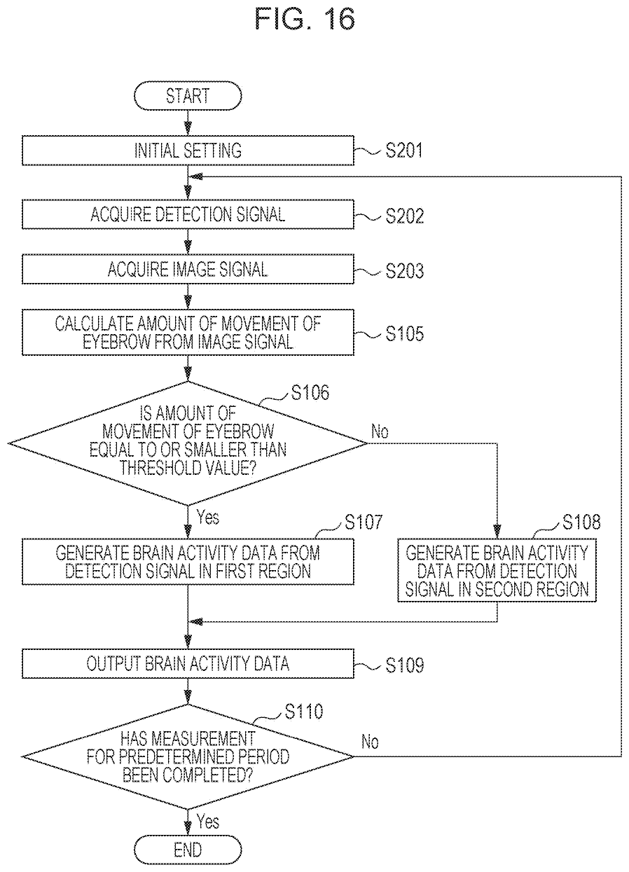

FIG. 16 is a flowchart illustrating an example of processing for generating brain activity data of the user 50. The flowchart illustrated in FIG. 16 is identical to the flowchart illustrated in FIG. 10 except for that steps S201 to S203 are provided instead of steps S101 to S104. Processes in steps S105 to S110 are identical to corresponding processes illustrated in FIG. 10.

In step S201, the processing circuit 234 performs initial setting necessary for measurement. The initial setting in the present embodiment includes a step of deciding a position of a feature point of the eyebrow of the user 50 in an initial state on the basis of an image signal output from the camera 270 and recording the position in the memory 236.

In step S202, the control circuit 232 turns the light sources 252 on and causes the photodetectors 254 to detect internal scattering light in corresponding regions. Each of the photodetectors 254 outputs a detection signal according to an intensity of the detected internal scattering light. The processing circuit 234 acquires the detection signal output from each of the photodetectors 254.

In step S203, the processing circuit 234 acquires an image signal output from the camera 270. Step S202 and step S203 may be performed in a reverse order or may be performed concurrently.

Processes in step S105 and the subsequent steps are identical to those in corresponding steps in the flowchart illustrated in FIG. 10, and description thereof is omitted.

Also in the present embodiment, in a case where an amount of movement of the eyebrow of the user 50 detected from an image signal is equal to or smaller than a threshold value, brain activity data is generated from a detection signal in the first region, and in a case where the amount of movement of the eyebrow is larger than the threshold value, brain activity data is generated from a detection signal in the second region. By such an operation, brain activity data based on a detection signal in an appropriate region can be output in accordance with an amount of change in facial expression of the user 50.

Although a single target region is selected from among the first and second regions in the above example, one or more target regions may be selected from among three or more regions, and brain activity data may be generated on the basis of a detection signal in the selected target regions.

Although a change in shape of skin of the forehead of the user 50 is detected on the basis of an image signal generated by the camera 270, which is a device different from the NIRS sensor 250, in the present embodiment, the change in shape of skin may be detected by another method. For example, another sensor that detects a change in shape of skin of the forehead of the user 50 and outputs a signal indicative of an amount of the change may be used. Alternatively, a sensor that detects a change in another physical amount that influences brain activity data instead of a change in shape of skin may be used. To “influence brain activity data” encompasses inclusion of noise in brain activity data and making brain activity data unmeasurable.

Also in the present embodiment, the target region may be selected on the basis of a temporal change of a cerebral blood flow signal instead of using an image signal. For example, the processing circuit 234 may decide the target region on the basis of a rate of change of a cerebral blood flow signal in the first region. For example, the processing circuit 234 may stop output of brain activity data based on a detection signal in the first region and output brain activity data based on a detection signal in the second region located on an upper side in a case where an absolute value of a rate of change of a cerebral blood flow signal is larger than a threshold value. By such an operation, the measurement device 200 can output more accurate brain activity data while maintaining sensitivity of measurement.

The processing circuit 234 may determine effectiveness of a detection signal in the first region on the basis of a phase of a change in oxyhemoglobin concentration and a phase of a change in deoxyhemoglobin concentration, as in the example illustrated in FIG. 13. Light sources that emit two kinds of light of different wavelengths may be used to effectively measure amounts of change in oxyhemoglobin concentration and deoxyhemoglobin concentration from reference values. For example, the NIRS sensor 250 may include a first light source that emits a first light pulse having a first wavelength that is equal to or longer than 650 nm and shorter than 805 nm in air and a second light source that emits a second light pulse having a second wavelength that is longer than 805 nm and equal to or shorter than 950 nm in air. In this case, the processing circuit 234 can generate a cerebral blood flow signal indicative of an oxyhemoglobin concentration and a deoxyhemoglobin concentration on the basis of detection signals output from two photodetectors that detect scattering light generated by light emitted from the first light source and scattering light generated by light emitted from the second light source, respectively.

The light emitted by the first light source and the second light source is not limited to pulsed light. The light emitted by the first light source and the second light source may be continuous light that oscillates continuously.

The processing circuit 134 may output brain activity data based on a detection signal in the first region in a case where a phase of a change in oxyhemoglobin concentration and a phase of a change in deoxyhemoglobin in the first region are reverse to each other and stop output of the brain activity data in the first region and output brain activity data based on a detection signal in the second region in a case where the phases are identical. Even by such an operation, it is possible to output more accurate brain activity data while maintaining sensitivity of measurement.

Various modifications described in the first embodiment are also applicable to the present embodiment.

Various modifications of the embodiments which a person skilled in the art can think of or any combinations of constituent elements and functions in the embodiments are also encompassed within the present disclosure without departing from the spirit of the present disclosure.

According to the technique of the present disclosure, information indicative of a state of brain activity of a user can be acquired. The technique of the present disclosure is applicable to various devices such as a camera, a measurement device, a smartphone, a tablet computer, and a head-mounted device.

What is claimed is:

1. A biological measurement device comprising:
a light emitting device that irradiates a first region and a second region of a forehead of a subject with light, the second region being located on an upper side relative to the first region;
a sensor that detects first scattering light generated by the light incident on the first region and second scattering light generated by the light incident on the second region and outputs detection signals according to intensities of the first scattering light and the second scattering light; and
a processing circuit that selects one of the first region and the second region as a target region on a basis of the detection signals and/or an image signal indicative of an image including a face of the subject and generates and outputs brain activity data indicative of a state of brain activity of the subject on a basis of the detection signal in the selected target region.

2. The biological measurement device according to claim 1, wherein
the sensor is an image sensor that outputs the detection signals and the image signal; and
the processing circuit selects the target region on a basis of a temporal change of the image signal.

3. The biological measurement device according to claim 1, further comprising an image sensor that outputs the image signal,
wherein the processing circuit selects the target region on a basis of a temporal change of the image signal.

4. The biological measurement device according to claim 1, wherein
the processing circuit detects movement of an eyebrow of the subject included in the image on a basis of a temporal change of the image signal and selects the target region on a basis of the movement of the eyebrow.

5. The biological measurement device according to claim 4, wherein
the processing circuit selects the second region as the target region in a case where an amount of displacement of the eyebrow of the subject from a reference position is larger than a threshold value during a measurement period and selects the first region as the target region in a case where the amount of displacement of the eyebrow of the subject from the reference position is not larger than the threshold value during the measurement period.

6. The biological measurement device according to claim 1, wherein
the processing circuit selects the target region on a basis of the detection signals output from the sensor.

7. The biological measurement device according to claim 6, wherein
the processing circuit generates a cerebral blood flow signal indicative of a state of hemoglobin in cerebral blood in the first region on a basis of the detection signal and selects the target region on a basis of a temporal change of the cerebral blood flow signal.

8. The biological measurement device according to claim 6, wherein
the processing circuit generates a cerebral blood flow signal indicative of temporal changes in oxyhemoglobin concentration and deoxyhemoglobin concentration in cerebral blood in the first region on a basis of the detection signal; and
the processing circuit selects the first region as the target region during a period where one of the oxyhemoglobin concentration and the deoxyhemoglobin concentration in the first region increases and an other one of the oxyhemoglobin concentration and the deoxyhemoglobin concentration in the first region decreases and selects the second region as the target region during a period where both of the oxyhemoglobin concentration and the deoxyhemoglobin concentration in the first region increase or decrease.

9. The biological measurement device according to claim 1, wherein
the processing circuit generates a cerebral blood flow signal indicative of a state of hemoglobin in cerebral blood in the first region and a cerebral blood flow signal indicative of a state of hemoglobin in cerebral blood in the second region on a basis of the detection signals in the first region and the second region and generates the brain activity data on a basis of the cerebral blood flow signal in the selected target region.

10. The biological measurement device according to claim 1, wherein
a part of the first region and a part of the second region overlap each other.

11. The biological measurement device according to claim 1, wherein
an area of the second region is larger than an area of the first region.

12. The biological measurement device according to claim 1, wherein
the light emitting device emits a light pulse toward the first region and the second region; and
the sensor detects the first scattering light and the second scattering light by detecting at least a part of a component after start of decrease in intensity among components of a reflected light pulse from the first region and the second region generated by emission of the light pulse.

13. The biological measurement device according to claim 1, wherein
the light emitting device includes a first light source that emits first irradiation light having a first wavelength that is equal to or longer than 650 nm and shorter than 805 nm in air and a second light source that emits second irradiation light having a second wavelength that is longer than 805 nm and equal to or shorter than 950 nm in air;
the sensor detects reflected light generated by irradiation of the first region with the first irradiation light and outputs a first detection signal according to an amount of the detected light, detects reflected light generated by irradiation of the first region with the second irradiation light and outputs a second detection signal according to an amount of the detected light, detects reflected light generated by irradiation of the second region with the first irradiation light and outputs a third detection signal according to an amount of the detected light, and detects reflected light generated by irradiation of the second region with the second irradiation light and outputs a fourth detection signal according to an amount of the detected light; and
the processing circuit generates a first cerebral blood flow signal indicative of a state of hemoglobin in a cerebral blood flow in the first region on a basis of the first detection signal and the second detection signal, generates a second cerebral blood flow signal indicative of a state of hemoglobin in a cerebral blood flow in the second region on a basis of the third detection signal and the fourth detection signal, and generates the brain activity data on a basis of the first cerebral blood flow signal or the second cerebral blood flow signal.

14. The biological measurement device according to claim 1, wherein
the processing circuit selects the first region as the target region and repeats an operation of generating and outputting the brain activity data on a basis of the detection signal in the first region; and
only in a case where the detection signal in the first region and/or the image signal satisfies a predetermined condition, the processing circuit selects the second region as the target region instead of the first region and generates the brain activity data on a basis of the detection signal in the second region.

15. A biological measurement method comprising:
irradiating a first region and a second region of a forehead of a subject with light, the second region being located on an upper side relative to the first region, detecting first scattering light generated by the light incident on the first region and second scattering light generated by the light incident on the second region, and acquiring detection signals according to intensities of the first scattering light and the second scattering light from a device that outputs the detection signals;
selecting one of the first region and the second region as a target region on a basis of the detection signals and/or an image signal indicative of an image including a face of the subject; and
generating and outputting brain activity data indicative of a state of brain activity of the subject on a basis of the detection signal in the selected target region.

16. A non-transitory computer-readable recording medium storing a program causing a computer to:
irradiate a first region and a second region of a forehead of a subject with light, the second region being located on an upper side relative to the first region, detect first scattering light generated by the light incident on the first region and second scattering light generated by the light incident on the second region, and acquire detection signals according to intensities of the first scattering light and the second scattering light from a device that outputs the detection signals;
select one of the first region and the second region as a target region on a basis of the detection signals and/or an image signal indicative of an image including a face of the subject; and
generate and output brain activity data indicative of a state of brain activity of the subject on a basis of the detection signal in the selected target region.

17. A biological measurement device comprising:
a light emitting device that outputs light including first light with which a first region of a forehead of a subject is irradiated and second light with which a second region located on an upper side relative to the first region is irradiated;
a sensor that detects first scattering light output from the subject on a basis of the first light, detects second scattering light output from the subject on a basis of the second light, outputs a first detection signal according to an intensity of the first scattering light, and outputs a second detection signal according to an intensity of the second scattering light; and
a processing circuit,
wherein the processing circuit determines whether or not the first region is suitable as a target region for measurement of a cerebral blood flow of the subject on a basis of (i) the first detection signal, (ii) an image signal indicative of an image including a face of the subject, or (iii) the first detection signal and the image signal, and generates and outputs brain activity data indicative of a state of brain activity of the subject on a basis of the first scattering light in a case where it is determined that the first region is suitable as the target region.

18. The biological measurement device according to claim 17, wherein
the processing circuit generates and outputs the brain activity data on a basis of the second scattering light in a case where it is determined that the first region is not suitable as the target region.

19. The biological measurement device according to claim 18, wherein
the processing circuit detects movement of an eyebrow of the subject included in the image on a basis of a temporal change of the image signal and determines whether or not the first region is suitable as the target region on a basis of the movement of the eyebrow.

20. The biological measurement device according to claim 19, wherein
the processing circuit determines that the first region is not suitable as the target region in a case where an amount of displacement of the eyebrow of the subject from a reference position is equal to or larger than a threshold value and determines that the first region is suitable as the target region in a case where the amount of displacement of the eyebrow of the subject from the reference position is smaller than the threshold value.

21. The biological measurement device according to claim 18, wherein
the processing circuit determines that the first region is not suitable as the target region, and determines again whether or not the first region is suitable as the target region during a period where the brain activity data is generated on a basis of the second scattering light; and
in a case where it is determined that the first region is suitable as the target region, the processing circuit changes the target region from the second region to the first region and generates the brain activity data on a basis of the first scattering light.

22. The biological measurement device according to claim 17, wherein
a part of the first region and a part of the second region overlap each other.

23. The biological measurement device according to claim 17, wherein
an area of the second region is larger than an area of the first region.

24. A biological measurement method comprising:
causing a light emitting device to output light including first light with which a first region of a forehead of a subject is irradiated and second light with which a second region located on an upper side relative to the first region is irradiated;
causing a sensor to detect first scattering light based on the first light, detect second scattering light based on the second light, output a first detection signal according to an intensity of the first scattering light, and output a second detection signal according to an intensity of the second scattering light;
determining whether or not the first region is suitable as a target region for measurement of a cerebral blood flow of the subject on a basis of (i) the first detection signal, (ii) an image signal indicative of an image including a face of the subject, or (iii) the first detection signal and the image signal; and
generating and outputting brain activity data indicative of a state of brain activity of the subject on a basis of the first scattering light in a case where it is determined that the first region is suitable as the target region.
